SEQUENCE LISTING (17 sequences)

SEQ ID NO: 1 - DNA, 19 bp, Artificial Sequence
  Chemically synthesized
acaucaccaaugcaauguu

SEQ ID NO: 2 - DNA, 19 bp, Artificial Sequence
  Chemically synthesized
gtacttcgccttcctcttc

SEQ ID NO: 3 - DNA, 21 bp, Artificial Sequence
  Chemically synthesized
aatggttacagtgagcaagaa

SEQ ID NO: 4 - DNA, 20 bp, Artificial Sequence
  Chemically synthesized
gattactgctctggctccta

SEQ ID NO: 5 - DNA, 20 bp, Artificial Sequence
  Chemically synthesized
atcgtactcctgcttgctga

SEQ ID NO: 6 - DNA, 20 bp, Artificial Sequence
  Chemically synthesized
cttctcattcctgcttgtgg

SEQ ID NO: 7 - DNA, 20 bp, Artificial Sequence
  Chemically synthesized
atctgagtgtgagggtctgg

SEQ ID NO: 8 - DNA, 19 bp, Artificial Sequence
  Chemically synthesized
agcaacagcaaggcgaaaa

SEQ ID NO: 9 - DNA, 20 bp, Artificial Sequence
  Chemically synthesized
ctggacctgtgggttgttga

SEQ ID NO: 10 - DNA, 21 bp, Artificial Sequence
  Chemically synthesized
agcaatgagacgatgaggctt

SEQ ID NO: 11 - DNA, 20 bp, Artificial Sequence
  Chemically synthesized
gcatttccacagtaccccca

SEQ ID NO: 12 - DNA, 20 bp, Artificial Sequence
  Chemically synthesized
acaagaccagactcccctgt

SEQ ID NO: 13 - DNA, 20 bp, Artificial Sequence
  Chemically synthesized
tctgggtcctgtagatggca

SEQ ID NO: 14 - DNA, 20 bp, Artificial Sequence
  Chemically synthesized
aaggcagtggagcaggtgaa

SEQ ID NO: 15 - DNA, 20 bp, Artificial Sequence
  Chemically synthesized
ccagcagactcaatacacac

SEQ ID NO: 16 - DNA, 20 bp, Artificial Sequence
  Chemically synthesized
agagaagaactgctgtgtgc

SEQ ID NO: 17 - DNA, 20 bp, Artificial Sequence
  Chemically synthesized
gggttgtgttggttgtagag

(19) United States
(12) Patent Application Publication
(10) Pub. No.: US 2020/0277404 A1
(43) Pub. Date: Sep. 3, 2020

(54) COMPOSITIONS AND METHODS FOR REGULATION OF IMMUNE CELL ACTIVATION AND PROLIFERATION

(71) Applicant: MONELL CHEMICAL SENSES CENTER, Philadelphia, PA (US)

(72) Inventors: Hong Wang, Havertown, PA (US); Pu Feng, Maple Glen, PA (US); Liquan Huang, Havertown, PA (US)

(21) Appl. No.: 16/645,597

(22) PCT Filed: Sep. 12, 2018
(86) PCT No.: PCT/US18/50581

§ 371 Date: Mar. 9, 2020

Related U.S. Application Data
(60) Provisional application No. 62/557,301, filed on Sep. 12, 2017.

(51) Int. Cl. C07K 16/40; C07K 16/28; C12N 15/113

(52) U.S. Cl. CPC C07K 16/40; C07K 16/28; C12N 15/1137; C12N 2310/12; C07K 2317/76; C12N 2310/11; C12N 15/1138

(57) ABSTRACT

The invention provides compositions and methods for regulating immune cell activation and/or proliferation by modulating a taste receptor type 1, a taste receptor type 2, Gustducin or TrpM5. In one embodiment, an activator of a taste receptor type 1, a taste receptor type 2, Gustducin or TrpM5 inhibits immune cell activation and/or proliferation. In one embodiment, an inhibitor of a taste receptor type 1, a taste receptor type 2, Gustducin or TrpM5 stimulates immune cell activation and/or proliferation. Thus, in diseases and conditions where a modulation of immune cell activation and/or proliferation is beneficial, modulators of a taste receptor type 1, a taste receptor type 2, Gustducin or TrpM5 act as therapeutics.

### CROSS-REFERENCE TO RELATED APPLICATIONS

This application claims priority from U.S. Provisional Application Ser. No. 62/557,301, filed on Sep. 12, 2017, which is incorporated by reference herein in its entirety.

### BACKGROUND OF THE INVENTION

Dysregulation of immune cell activation and proliferation contributes to various diseases. In inflammatory, autoimmune, and autoinflammatory diseases, various types of immune cells are over activated and attack host tissues and organs, which results in tissue and organ damage and thus the development of diseases. In these types of ailments, downregulation of immune cell activation and proliferation is beneficial for patients. On the other hand, in some other diseases, such as cancer and chronic infections, cancer cells or pathogens can evade immune surveillance by inhibiting immune cell activation and proliferation, and therefore under these conditions, upregulation of immune cell activation and proliferation can stimulate immune cells to attack cancerous cells and pathogen-infected cells to control disease progression.

Various immune control mechanisms have been discovered, and therapies based on these mechanisms are being used to treat a variety of illnesses. However, very often, not all patients respond to the currently available treatments.

Accordingly, there exists a need for improved methods and compositions for regualting immune cell activation. The present meets this need.

### SUMMARY OF THE INVENTION

The present invention provides a composition for regulating immune cell activation. In one embodiment, the composition comprising a modulator of one selected from the group consisting of a taste receptor type 1 (T1R), a taste receptor type 2 (T2R), Gustducin (Gust), Transient receptor potential cation channel subfamily M member 5 (TrpM5) and a combination thereof.

In one embodiment, the modulator is an inhibitor of one selected from the group consisting of a T1R, a T2R, Gust, TrpM5 and a combination thereof. In one embodiment, the modulator is an activator of one selected from the group consisting of a T1R, a T2R, Gust, TrpM5 and a combination thereof. In one embodiment, the modulator is at least one of the group consisting of a chemical compound, a protein, a peptide, a peptidomemetic, an antibody, a ribozyme, a small molecule chemical compound, a nucleic acid, a vector, an antisense nucleic acid molecule.

In one embodiment, the T1R is selected from the group consisting of T1R1, T1R2, and T1R3. In one embodiment, the T1R is T1R3. In one embodiment, the T2R is selected from the group consisting of T2R1, T2R2, T2R3, T2R4, T2R5, T2R6, T2R7, T2R8, T2R9, T2R10, T2R11, T2R12, T2R13, T2R14, T2R15, T2R16, T2R17, T2R18, T2R19, T2R20, T2R21, T2R22, T2R24, T2R25, T2R27, T2R28, T2R29, T2R30, T2R31, T2R32, T2R33, T2R34, T2R35, T2R36, T2R37, T2R38, T2R39, T2R40, T2R41, T2R42, T2R43, T2R45, T2R46, T2R50, T2R52, T2R53, T2R54, T2R55, T2R56, T2R57, T2R58, T2R59, T2R60, T2R62P, T2R63P, T2R64P, T2R23, T2R48, T2R49, T2R26, T2R47, T2R44, and T2R51.

The invention also provides a method for treating a disease or disorder associated with abnormal immune cell activation. In one embodiment, the method comprising administering a modulator of one selected from the group consisting of a taste receptor type 1 (T1R), a taste receptor type 2 (T2R), Gustducin (Gust), Transient receptor potential cation channel subfamily M member 5 (TrpM5) and a combination thereof to a subject in need thereof.

In one embodiment, the disease or disorder is associated with overactive immune cell activation. In one embodiment, the disease or disorder is selected from the group consisting of an inflammatory disease or disorder, an autoimmune disease or disorder, an autoinflammatory disease or disorder, a disease or disorder associated with inflammation, and a disease or disorder associated with immune cell activation. In one embodiment, the disease or disorder is selected from the group consisting of cancer and a chronic infectious disease or disorder.

In one embodiment, the modulator is an activator of one selected from the group consisting of a T1R, a T2R, Gust, TrpM5 and a combination thereof. In one embodiment, the modulator is an inhibitor of one selected from the group consisting of a T1R, a T2R, Gust, TrpM5 and a combination thereof.

In one embodiment, the T1R is selected from the group consisting of T1R1, T1R2, and T1R3. In one embodiment, the T1R is T1R3. In one embodiment, the T2R is selected from the group consisting of T2R1, T2R2, T2R3, T2R4, T2R5, T2R6, T2R7, T2R8, T2R9, T2R10, T2R11, T2R12, T2R13, T2R14, T2R15, T2R16, T2R17, T2R18, T2R19, T2R20, T2R21, T2R22, T2R24, T2R25, T2R27, T2R28, T2R29, T2R30, T2R31, T2R32, T2R33, T2R34, T2R35, T2R36, T2R37, T2R38, T2R39, T2R40, T2R41, T2R42, T2R43, T2R45, T2R46, T2R50, T2R52, T2R53, T2R54, T2R55, T2R56, T2R57, T2R58, T2R59, T2R60, T2R62P, T2R63P, T2R64P, T2R23, T2R48, T2R49, T2R26, T2R47, T2R44, and T2R51.

The invention also provides a method of regulating immune cell activation and/or proliferation. In one embodiment, the method comprising administering a modulator of one selected from the group consisting of a taste receptor type 1 (T1R), a taste receptor type 2 (T2R), Gustducin (Gust), Transient receptor potential cation channel subfamily M member 5 (TrpM5) and a combination thereof to a subject in need thereof.

In one embodiment, the method decreases immune cell activation and/or proliferation, and wherein the modulator is an activator of one selected from the group consisting of a T1R, a T2R, Gust, TrpM5 and a combination thereof. In one embodiment, the method increases immune cell activation and/or proliferation, and wherein the modulator is an inhibitor of one selected from the group consisting of a T1R, a T2R, Gust, TrpM5 and a combination thereof.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1, comprising FIG. 1A and FIG. 1B, depicts experimental results demonstrating the expression of T1R3, Gustducin (Gust) and Transient receptor potential cation channel subfamily M member 5 (TrpM5) in mouse immune organs and structures. FIG. 1A depicts RT-PCR analysis of Gust and T1R3 mRNA expression in intestine, tongue, thymus and spleen tissues. FIG. 1B depicts immunofluorescent staining using antibodies to T1R3, Gust, TrpM5, CD3 (a T cell marker) and CD11b (a monocyte/macrophage/neutrophil marker).

FIG. 2, comprising FIG. 2A and FIG. 2B, depicts experimental results demonstrating the expression of taste receptors, Gust, TrpM5, and PLC-β2 in EL-4 mouse T-cell line. FIG. 2A depicts RT-PCR analysis of mRNA expression. RT+ and RT− represent reverse transcription reactions with or without reverse transcriptase. FIG. 2B depicts immunofluorescent staining of EL-4 cells using antibodies to T2R16, Gust and PLC-β2. Control antibody is nonspecific rabbit IgG.

FIG. 3, comprising FIG. 3A through FIG. 3C, depicts experimental results demonstrating hyper-activation of immune cells (splenocytes) from Gust-knockout (Gust-KO), T1R3-knockout (T1R3-KO) or TrpM5-knockout (TrpM5-KO) mice. Splenocytes were treated with immune activators lipopolysaccharide (LPS), Convanavalin A (ConA) or an antibody to CD3. FIG. 3A depicts hyper-activation of immune cells isolated from Gust-KO mice. FIG. 3B depicts hyper-activation of immune cells isolated from T1R3-KO mice. FIG. 3C depicts hyper-activation of immune cells isolated from TrpM5-KO mice.

FIG. 4 depicts experimental results demonstrating that the functions of T2Rs can be strengthened by the Toll-like receptor and the glucocorticoid receptor pathways.

FIG. 5, comprising FIG. 5A through FIG. 5E, depicts experimental results demonstrating that α-Gustducin-knockout mice show aggravated colitis. Wild-type (WT) and α-gustducin-knockout (KO) mice were given 3% DSS in drinking water for 7 days.

FIG. 5A depicts the percentage of body weight loss: α-gustducin-knockout mice lost significantly more body weight than did wild-type mice. N=16-18 per group. FIG. 5B depicts the colitis disease index based on the severity of diarrhea and rectal bleeding: α-gustducin-knockout mice exhibited a higher disease index than did wild-type mice. FIG. 5C depicts colon (upper panel) and spleen (lower panels) from representative wild-type and α-gustducin-knockout mice 7 days after DSS administration. α-Gustducin-knockout mice had shorter colons and much enlarged spleens compared to wild-type mice. FIG. 5D depicts tissue injury scores based on histological staining of colon tissues from wild-type and α-gustducin-knockout mice 7 days after DSS administration. FIG. 5E depicts H&E staining of colon tissues from wild-type and α-gustducin-knockout mice not treated with DSS (water) or treated with DSS for 7 days (DSS). Without DSS treatment, colons from wild-type and α-gustducin-knockout mice showed normal morphology. After DSS administration, α-gustducin-knockout mice showed more pronounced tissue damage along the length of the colon compared to wild-type mice. *p<0.05, **p<0.005, ***p<0.0005; ANOVA with post hoc t-tests.

FIG. 6, comprising FIG. 6A through FIG. 6C, depicts experimental results demonstrating that α-Gustducin-knockout mice display increased inflammation in DSS-induced colitis. Wild-type (WT) and α-gustducin-knockout (KO) mice were given 3% DSS in drinking water for 7 days. FIG. 6A depicts results demonstrating massive immune cell infiltration in colon of DSS-treated α-gustducin-knockout mice. Distal colon tissues were processed for immunohistochemistry with antibodies specific to indicated immune cell types. FIG. 6B depicts corresponding immune cell numbers as the percentage of immunostained areas divided by the total area of tissue measured based on image analyses. FIG. 6C depicts results demonstrating that DSS-induced colitis in α-gustducin-knockout mice elicits increased expression of TNF and IFN-γ and decreased expression of IL-5, IL-13, and IL-10 in colon. Real-time quantitative RT-PCR was performed using gene-specific primers. β-Actin was used as the endogenous control gene. Gene expression levels in the colon of wild-type mice were designated as 1. N=5 per group. *p<0.05, **p<0.005; t-tests.

FIG. 7, comprising FIG. 7A through FIG. 7E, depicts experimental results demonstrating that there are increased inflammatory responses in α-gustducin knockout mice. FIG. 7A depicts results demonstrating that the spleen of DSS-treated α-gustducin knockout mice (KO) were significantly enlarged compared to DSS-treated wild-type mice (WT). FIG. 7B depicts results demonstrating that mean plasma levels of TNF were higher in DSS-treated α-gustducin knockout mice than in wild-type mice. FIG. 7C depicts results demonstrating that the mRNA levels of TNF were significantly higher in the colon of non-stimulated α-gustducin knockout mice than in the colon of non-stimulated wild-type mice. FIG. 7D depicts results demonstrating that the basal levels of secreted TNF from non-stimulated colon explants of wild-type mice were not significantly different from those of α-gustducin knockout mice. FIG. 7E depicts results demonstrating that lipopolysaccharide (LPS) stimulated higher levels of TNF secretion from colon explant culture (overnight) of α-gustducin knockout mice than from that of wild-type mice. MED: culture medium. *p<0.05. ***p<0.001.

### DETAILED DESCRIPTION OF THE INVENTION

The present invention relates to compositions and methods for modulating taste receptors, Gustducin, and/or TrpM5 to regulate immune cell activation. In one embodiment, invention relates to compositions and methods for modulating taste receptors, Gustducin, and/or TrpM5 to treat or prevent a disease or disorder associated with abnormal immune cell activation.

In one embodiment, the composition of the present invention comprises a modulator of one or more of Taste Receptors Type 1 (T1Rs), Taste Receptors Type 2 (T2Rs), Gustducin and TrpM5. For example, in one embodiment, the modulator of one or more of T1Rs, T2Rs, Gustducin and TrpM5 is an inhibitor of one or more of Taste Receptors Type 1 T1Rs, T2Rs, Gustducin and TrpM5, which inhibits the expression, activity, or both of one or more of T1Rs, T2Rs, Gustducin and TrpM5.

In one embodiment, the modulator of one or more of T1Rs, T2Rs, Gustducin and TrpM5 is an activator of one or more of T1Rs, T2Rs, Gustducin and TrpM5, which increases the expression, activity, or both of one or more of T1Rs, T2Rs, Gustducin and TrpM5. For example, in certain embodiments, the activator of one or more of T1Rs, T2Rs, Gustducin and TrpM5 increases the expression or activity of one or more of T1Rs, T2Rs, Gustducin and TrpM5.

In one embodiment, the method of the present invention comprises regulating immune cell activation. For example, in one embodiment, the method increases immune cell activation. In one embodiment, the method decreases immune cell activation. In one embodiment, the method comprises administering to a subject an effective amount of a composition comprising a modulator of one or more of T1Rs, T2Rs, Gustducin and TrpM5.

In one embodiment, the method of the present invention comprises treating or preventing a disease or disorder associated with abnormal immune cell activation. For example, in one embodiment, the method treats or prevents a disease or disorder associated overactive immune cell activation. In one embodiment, the method treats or prevents a disease or disorder associated down-regulated or low immune cell activation. In one embodiment, the method comprises administering a modulator of one or more of T1Rs, T2Rs, Gustducin and TrpM5 to the subject.

### Definitions

Unless defined otherwise, all technical and scientific terms used herein have the same meaning as commonly understood by one of ordinary skill in the art to which this invention belongs.

Generally, the nomenclature used herein and the laboratory procedures in cell culture, molecular genetics, organic chemistry, and nucleic acid chemistry and hybridization are those well-known and commonly employed in the art.

Standard techniques are used for nucleic acid and peptide synthesis. The techniques and procedures are generally performed according to conventional methods in the art and various general references (e.g., Sambrook and Russell, 2012, Molecular Cloning, A Laboratory Approach, Cold Spring Harbor Press, Cold Spring Harbor, N.Y., and Ausubel et al., 2012, Current Protocols in Molecular Biology, John Wiley & Sons, NY), which are provided throughout this document.

The nomenclature used herein and the laboratory procedures used in analytical chemistry and organic syntheses described below are those well-known and commonly employed in the art. Standard techniques or modifications thereof are used for chemical syntheses and chemical analyses.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, or ±10%, or ±5%, or ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The an antibody in the present invention may exist in a variety of forms where the antigen binding portion of the antibody is expressed as part of a contiguous polypeptide chain including, for example, a single domain antibody fragment (sdAb), a single chain antibody (scFv) and a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragment” refers to at least one portion of an intact antibody and refers to the antigenic determining variable regions of an intact antibody. Examples of antibody fragments include, but are not limited to, Fab, Fab′, F(ab′)2, and Fv fragments, linear antibodies, sdAb (either V_{L} or V_{H}), camelid V_{HH} domains, scFv antibodies, and multi-specific antibodies formed from antibody fragments. The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it was derived. Unless specified, as used herein an scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}.

An “antibody heavy chain,” as used herein, refers to the larger of the two types of polypeptide chains present in antibody molecules in their naturally occurring conformations, and which normally determines the class to which the antibody belongs.

An “antibody light chain,” as used herein, refers to the smaller of the two types of polypeptide chains present in antibody molecules in their naturally occurring conformations. Kappa (κ) and lambda (λ) light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art. The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition.

“Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

As used herein, an “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes.

As used herein, an “autoimmune disease” refers to a disease arising from an inappropriate immune response of the body of a subject against substances and tissues normally present in the body. This may be restricted to certain organs (e.g., in autoimmune thyroiditis) or involve a particular tissue in different places (e.g., Goodpasture's disease which may affect the basement membrane in both the lung and kidney).

An “autoinflammatory disease” refers to a category of diseases that are similar but different from autoimmune diseases. Autoinflammatory and autoimmune diseases share common characteristics in that both groups of disorders result from the immune system attacking a subject's own tissues and result in increased inflammation. In autoinflammatory diseases, a subject's innate immune system causes inflammation for unknown reasons. The innate immune system reacts even though it has never encountered autoantibodies or antigens in the subject. Autoinflammatory disorders are characterized by intense episodes of inflammation that result in such symptoms as fever, rash, or joint swelling. These diseases also carry the risk of amyloidosis, a potentially fatal buildup of a blood protein in vital organs.

An “effective amount” or “therapeutically effective amount” of a compound is that amount of a compound which is sufficient to provide a beneficial effect to the subject to which the compound is administered. An “effective amount” of a delivery vehicle is that amount sufficient to effectively bind or deliver a compound.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The terms “patient,” “subject,” “individual,” and the like are used interchangeably herein, and refer to any animal, or cells thereof whether in vitro or in vivo, amenable to the methods described herein. In certain non-limiting embodiments, the patient, subject or individual is a human.

A “therapeutic” treatment is a treatment administered to a subject who exhibits signs or symptoms of pathology, for the purpose of diminishing or eliminating those signs or symptoms.

The terms “treat,” “treating,” and “treatment,” refer to therapeutic or preventative measures described herein. The methods of “treatment” employ administration to a subject, in need of such treatment, a composition of the present invention, for example, a subject afflicted a disease or disorder, or a subject who ultimately may acquire such a disease or disorder, in order to prevent, cure, delay, reduce the severity of, or ameliorate one or more symptoms of the disorder or recurring disorder, or in order to prolong the survival of a subject beyond that expected in the absence of such treatment.

As used herein, “treating a disease or disorder” means reducing the severity and/or frequency with which a sign or symptom of the disease or disorder is experienced by a patient.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific.

In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of a mRNA molecule also consists of the nucleotide residues of the mRNA molecule which are matched with an anti-codon region of a transfer RNA molecule during translation of the mRNA molecule or which encode a stop codon. The coding region may thus include nucleotide residues comprising codons for amino acid residues which are not present in the mature protein encoded by the mRNA molecule (e.g., amino acid residues in a protein export signal sequence).

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In one embodiment, the first region comprises a first portion and the second region comprises a second portion, whereby, when the first and second portions are arranged in an antiparallel fashion, at least about 50%, at least about 75%, at least about 90%, or at least about 95% of the nucleotide residues of the first portion are capable of base pairing with nucleotide residues in the second portion. In one embodiment, all nucleotide residues of the first portion are capable of base pairing with nucleotide residues in the second portion.

The term “DNA” as used herein is defined as deoxyribonucleic acid.

The term “expression” as used herein is defined as the transcription and/or translation of a particular nucleotide sequence driven by its promoter.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

The term “fusion polypeptide” refers to a chimeric protein containing a protein of interest (e.g., luciferase) joined to a heterologous sequence (e.g., a non-luciferase amino acid or protein).

The term “homology” refers to a degree of complementarity. There may be partial homology or complete homology (i.e., identity). Homology is often measured using sequence analysis software (e.g., Sequence Analysis Software Package of the Genetics Computer Group. University of Wisconsin Biotechnology Center. 1710 University Avenue. Madison, Wis. 53705). Such software matches similar sequences by assigning degrees of homology to various substitutions, deletions, insertions, and other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

“Isolated” means altered or removed from the natural state. For example, a nucleic acid or a peptide naturally present in its normal context in a living animal is not “isolated,” but the same nucleic acid or peptide partially or completely separated from the coexisting materials of its natural context is “isolated.” An isolated nucleic acid or protein can exist in substantially purified form, or can exist in a non-native environment such as, for example, a host cell.

The term “isolated” when used in relation to a nucleic acid, as in “isolated oligonucleotide” or “isolated polynucleotide” refers to a nucleic acid sequence that is identified and separated from at least one contaminant with which it is ordinarily associated in its source. Thus, an isolated nucleic acid is present in a form or setting that is different from that in which it is found in nature. In contrast, non-isolated nucleic acids (e.g., DNA and RNA) are found in the state they exist in nature. For example, a given DNA sequence (e.g., a gene) is found on the host cell chromosome in proximity to neighboring genes; RNA sequences (e.g., a specific mRNA sequence encoding a specific protein), are found in the cell as a mixture with numerous other mRNAs that encode a multitude of proteins. However, isolated nucleic acid includes, by way of example, such nucleic acid in cells ordinarily expressing that nucleic acid where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide contains at a minimum, the sense or coding strand (i.e., the oligonucleotide may be single-stranded), but may contain both the sense and anti-sense strands (i.e., the oligonucleotide may be double-stranded).

The term “isolated” when used in relation to a polypeptide, as in “isolated protein” or “isolated polypeptide” refers to a polypeptide that is identified and separated from at least one contaminant with which it is ordinarily associated in its source. Thus, an isolated polypeptide is present in a form or setting that is different from that in which it is found in nature. In contrast, non-isolated polypeptides (e.g., proteins and enzymes) are found in the state they exist in nature.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). The term “nucleic acid” typically refers to large polynucleotides.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

By “expression cassette” is meant a nucleic acid molecule comprising a coding sequence operably linked to promoter/regulatory sequences necessary for transcription and, optionally, translation of the coding sequence.

The term “operably linked” as used herein refer to the linkage of nucleic acid sequences in such a manner that a nucleic acid molecule capable of directing the transcription of a given gene and/or the synthesis of a desired protein molecule is produced. The term also refers to the linkage of sequences encoding amino acids in such a manner that a functional (e.g., enzymatically active, capable of binding to a binding partner, capable of inhibiting, etc.) protein or polypeptide is produced.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a n inducible manner.

An “inducible” promoter is a nucleotide sequence which, when operably linked with a polynucleotide which encodes or specifies a gene product, causes the gene product to be produced substantially only when an inducer which corresponds to the promoter is present.

A “constitutive” promoter is a nucleotide sequence which, when operably linked with a polynucleotide which encodes or specifies a gene product, causes the gene product to be produced in a cell under most or all physiological conditions of the cell.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, a “peptidomimetic” is a compound containing non-peptidic structural elements that is capable of mimicking the biological action of a parent peptide. A peptidomimetic may or may not comprise peptide bonds.

The term “RNA” as used herein is defined as ribonucleic acid.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

The term “recombinant polypeptide” as used herein is defined as a polypeptide produced by using recombinant DNA methods.

As used herein, “conjugated” refers to covalent attachment of one molecule to a second molecule.

As used herein, the term “transdominant negative mutant gene” refers to a gene encoding a polypeptide or protein product that prevents other copies of the same gene or gene product, which have not been mutated (i.e., which have the wild-type sequence) from functioning properly (e.g., by inhibiting wild type protein function). The product of a transdominant negative mutant gene is referred to herein as “dominant negative” or “DN” (e.g., a dominant negative protein, or a DN protein).

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, such as a human.

The phrase “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

The phrase “activate,” as used herein, means to increase a molecule, a reaction, an interaction, a gene, an mRNA, and/or a protein's expression, stability, function or activity by a measurable amount. Activators are compounds that, e.g., bind to, increase stimulation, activation, activate, sensitize, or upregulate a protein, a gene, and an mRNA stability, expression, function and activity, e.g., agonists.

“Test agents,” “test compounds,” or “test compositions” as used herein refers to an agent, composition or compound that is to be screened in one or more of the assays described herein. Test agents include compounds of a variety of general types including, but not limited to, small organic molecules, known pharmaceuticals, polypeptides; carbohydrates such as oligosaccharides and polysaccharides; polynucleotides; lipids or phospholipids; fatty acids; steroids; or amino acid analogs. Test agents can be obtained from libraries, such as natural product libraries and combinatorial libraries. In addition, methods of automating assays are known that permit screening of several thousands of compounds in a short period.

“Variant” as the term is used herein, is a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based, in part, on the unexpected discovery of a novel method of regulating immune cell activation by modulating one or more of T1Rs, T2Rs, Gustducin and TrpM5. In some embodiments, modulators of one or more of T1Rs, T2Rs, Gustducin and TrpM5 regulation of immune cell activation thereby treating or preventing diseases and disorders associated abnormal immune cell activation. Accordingly, the invention provides compositions and methods for modulating the level of or activity of T1Rs, T2Rs, Gustducin and TrpM5.

In one embodiment, the immune cells includes but is not limited to T cells, B cells, natural killer (NK) cells, natural killer T (NKT) cells, mast cells, myeloid-derived phagocytes, and the like. However, the invention should not be limited to any specific immune cell.

### Taste Receptors

G-protein-coupled taste receptors, T1Rs and T2Rs, and their downstream signaling molecules, such as Gustducin and TrpM5, were discovered from taste bud cells in the oral cavity.

The T1Rs belong to the dimeric Class III GPCRs, with large N-terminal extracellular domains, which forms a Venus Flytrap structure. T1R family members include T1R1, T1R2, and T1R3, e.g., rT1R3 (encoded by rat Tas1r3 gene), mT1R3 (encoded by mouse Tas1r3 gene), hT1R3 (encoded by human TAS1R3 gene), rT1R2 (encoded by rat Tas1r2 gene), mT1R2 (encoded by mouse Tas1r2 gene), hT1R2 (encoded by human TAS1R2 gene), and rT1R1 (encoded by rat Tas1r1 gene), mT1R1 (encoded by mouse Tas1r1 gene) and hT1R1 (encoded by human TAS1R1 gene), any functional polymorphisms thereof and any isoforms derived by alternative splicing of the mRNAs encoded by the same genes.

T2Rs resemble Class I GPCRs with binding sites in the transmembrane helices. T2R family members include T2R1, T2R2, T2R3, T2R4, T2R5, T2R6, T2R7, T2R8, T2R9, T2R10, T2R11, T2R12, T2R13, T2R14, T2R15, T2R16, T2R17, T2R18, T2R19, T2R20, T2R21, T2R22, T2R24, T2R25, T2R27, T2R28, T2R29, T2R30, T2R31, T2R32, T2R33, T2R34, T2R35, T2R36, T2R37, T2R38, T2R39, T2R40, T2R41, T2R42, T2R43, T2R45, T2R46, T2R50, T2R52, T2R53, T2R54, T2R55, T2R56, T2R57, T2R58, T2R59, T2R60, T2R62P, T2R63P, T2R64P, T2R23, T2R48, T2R49, T2R26, T2R47, T2R44, and T2R51.

### Inhibitors

In one embodiment, the present invention provides a composition for regulating immune cell activation. In one embodiment, the present invention provides a composition for treating or preventing a disease or disorder associated with abnormal immune cell activation. In certain embodiments, the composition inhibits the expression, activity, or both of a T1R, a T2R, Gustducin, TrpM5, or a combination thereof of the subject.

In one embodiment, the composition of the invention comprises an inhibitor of a T1R, a T2R, Gustducin, or TrpM5. An inhibitor of a T1R, a T2R, Gustducin, or TrpM5 is any compound, molecule, or agent that reduces, inhibits, or prevents the function of a T1R, a T2R, Gustducin, or TrpM5. For example, an inhibitor of a T1R, a T2R, Gustducin, or TrpM5 is any compound, molecule, or agent that reduces expression, activity, or both of a T1R, a T2R, Gustducin, or TrpM5. In one embodiment, an inhibitor of a T1R, a T2R, Gustducin, or TrpM5 comprises a nucleic acid, a peptide, a small molecule, a siRNA, a ribozyme, an antisense nucleic acid, an antagonist, an aptamer, a peptidomimetic, or any combination thereof.

In one embodiment, the composition comprises an inhibitor of T1R1, T1R2, T1R3, T2R1, T2R2, T2R3, T2R4, T2R5, T2R6, T2R7, T2R8, T2R9, T2R10, T2R11, T2R12, T2R13, T2R14, T2R15, T2R16, T2R17, T2R18, T2R19, T2R20, T2R21, T2R22, T2R24, T2R25, T2R27, T2R28, T2R29, T2R30, T2R31, T2R32, T2R33, T2R34, T2R35, T2R36, T2R37, T2R38, T2R39, T2R40, T2R41, T2R42, T2R43, T2R45, T2R46, T2R50, T2R52, T2R53, T2R54, T2R55, T2R56, T2R57, T2R58, T2R59, T2R60, T2R62P, T2R63P, T2R64P, T2R23, T2R48, T2R49, T2R26, T2R47, T2R44, T2R51, Gustudcin or TrpM5.

For example, in one embodiment, inhibitors of T1Rs, T2Rs, Gustducin or TrpM5 include, but are not limited to, Lactisole (Sodium 2-(4-methoxyphenoxy)propanoate), Probenecid (p-(Dipropylsulfamoyl)benzoic acid), TPPO (triphenylphosphine oxide), a T1R targeting shRNA, a TrpM5 targeting siRNA/shRNA, and a Gustducin targeting siRNA/shRNA.

In one embodiment, T1R inhibitors include Lactisole and a T1R targeting siRNA/shRNA. In one embodiment, the T1R shRNA comprises the sequence ACAUCACCAAUGCAAUGUU (SEQ ID NO:1)

In one embodiment, T2R inhibitors include Probenecid (p-(Dipropylsulfamoyl)benzoic acid) and a T2R targeting siRNA/shRNA.

In one embodiment, TrpM5 inhibitors include TPPO, and a TrpM5 targeting siRNA/shRNA. In one embodiment, the TrpM5 targeting siRNA/shRNA comprises the sequence GTACTTCGCCTTCCTCTTC (SEQ ID NO:2)

In one embodiment, Gustducin inhibitors include a Gustducin targeting siRNA/shRNA. In one embodiment, the Gustducin targeting siRNA comprises the sequence AATGGTTACAGTGAGCAAGAA (SEQ ID NO:3).

Small Molecule Inhibitors

In various embodiments, the inhibitor is a small molecule. When the inhibitor is a small molecule, a small molecule may be obtained using standard methods known to the skilled artisan. Such methods include chemical organic synthesis or biological means. Biological means include purification from a biological source, recombinant synthesis and in vitro translation systems, using methods well known in the art. In one embodiment, a small molecule inhibitor of the invention comprises an organic molecule, inorganic molecule, biomolecule, synthetic molecule, and the like.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

The small molecule and small molecule compounds described herein may be present as salts even if salts are not depicted and it is understood that the invention embraces all salts and solvates of the inhibitors depicted here, as well as the non-salt and non-solvate form of the inhibitors, as is well understood by the skilled artisan. In some embodiments, the salts of the inhibitors of the invention are pharmaceutically acceptable salts.

Where tautomeric forms may be present for any of the inhibitors described herein, each and every tautomeric form is intended to be included in the present invention, even though only one or some of the tautomeric forms may be explicitly depicted. For example, when a 2-hydroxypyridyl moiety is depicted, the corresponding 2-pyridone tautomer is also intended.

The invention also includes any or all of the stereochemical forms, including any enantiomeric or diasteriomeric forms of the inhibitors described. The recitation of the structure or name herein is intended to embrace all possible stereoisomers of inhibitors depicted. All forms of the inhibitors are also embraced by the invention, such as crystalline or non-crystalline forms of the inhibitors. Compositions comprising an inhibitor of the invention are also intended, such as a composition of substantially pure inhibitor, including a specific stereochemical form thereof, or a composition comprising mixtures of inhibitors of the invention in any ratio, including two or more stereochemical forms, such as in a racemic or non-racemic mixture.

In one embodiment, the small molecule inhibitor of the invention comprises an analog or derivative of an inhibitor described herein.

In one embodiment, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule inhibitors described herein are derivatized/analoged as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be homocycles or heterocycles.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule inhibitors described herein or can be based on a scaffold of a small molecule inhibitor described herein, but differing from it in respect to certain components or structural makeup, which may have a similar or opposite action metabolically. An analog or derivative of any of a small molecule inhibitor in accordance with the present invention can be used to reduce skin pigmentation.

In one embodiment, the small molecule inhibitors described herein can independently be derivatized/analoged by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

Nucleic Acid Inhibitors

In other related aspects, the invention includes an isolated nucleic acid. In some instances, the inhibitor is an siRNA, shRNA or antisense molecule, which inhibits a T1R, a T2R, Gustducin or TrpM5. In one embodiment, the nucleic acid comprises a promoter/regulatory sequence such that the nucleic acid is capable of directing expression of the nucleic acid. Thus, the invention encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In another aspect of the invention, a T1R, a T2R, Gustducin or TrpM5, can be inhibited by way of inactivating and/or sequestering a T1R, a T2R, Gustducin or TrpM5. As such, inhibiting the activity of a T1R, a T2R, Gustducin or TrpM5 can be accomplished by using a transdominant negative mutant.

In one embodiment, siRNA or shRNA is used to decrease the level of a T1R, a T2R, Gustducin or TrpM5 protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, P A (2003); and Gregory J. Hannon, Ed., RNAi A Guide to Gene Silencing, Cold Spring Harbor Laboratory Press, Cold Spring Harbor, N.Y. (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of a T1R, a T2R, Gustducin or TrpM5 using RNAi technology.

In another aspect, the invention includes a vector comprising an siRNA or antisense polynucleotide. In one embodiment, the siRNA or antisense polynucleotide is capable of inhibiting the expression of a target polypeptide, wherein the target polypeptide is selected from the group consisting of p21 and telomerase. The incorporation of a desired polynucleotide into a vector and the choice of vectors is well-known in the art as described in, for example, Sambrook et al. (2012), and in Ausubel et al. (1997), and elsewhere herein.

In certain embodiments, the expression vectors described herein encode a short hairpin RNA (shRNA) inhibitor. shRNA inhibitors are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleaves the shRNA to form siRNA.

In one embodiment, the nucleic acid inhibitor of a T1R comprises the sequence ACAUCACCAAUGCAAUGUU (SEQ ID NO:1).

In one embodiment, the nucleic acid inhibitor of TrpM5 comprises the sequence GTACTTCGCCTTCCTCTTC (SEQ ID NO:2).

In one embodiment, the nucleic acid inhibitor of Gustudcin comprises the sequence AATGGTTACAGTGAGCAAGAA (SEQ ID NO:3).

The siRNA, shRNA, or antisense polynucleotide can be cloned into a number of types of vectors as described elsewhere herein. For expression of the siRNA or antisense polynucleotide, at least one module in each promoter functions to position the start site for RNA synthesis.

In order to assess the expression of the siRNA, shRNA, or antisense polynucleotide, the expression vector to be introduced into a cell can also contain either a selectable marker gene or a reporter gene or both to facilitate identification and selection of expressing cells from the population of cells sought to be transfected or infected using a viral vector. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

Therefore, in another aspect, the invention relates to a vector, comprising the nucleotide sequence of the invention or the construct of the invention. The choice of the vector will depend on the host cell in which it is to be subsequently introduced. In a particular embodiment, the vector of the invention is an expression vector. Suitable host cells include a wide variety of prokaryotic and eukaryotic host cells. In specific embodiments, the expression vector is selected from the group consisting of a viral vector, a bacterial vector and a mammalian cell vector. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2012), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

By way of illustration, the vector in which the nucleic acid sequence is introduced can be a plasmid which is or is not integrated in the genome of a host cell when it is introduced in the cell. Illustrative, non-limiting examples of vectors in which the nucleotide sequence of the invention or the gene construct of the invention can be inserted include a tet-on inducible vector for expression in eukaryote cells.

The vector may be obtained by conventional methods known by persons skilled in the art (Sambrook et al., 2012). In a particular embodiment, the vector is a vector useful for transforming animal cells.

In one embodiment, the recombinant expression vectors may also contain nucleic acid molecules which encode a peptide or peptidomimetic inhibitor of invention, described elsewhere herein.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The recombinant expression vectors may also contain a selectable marker gene which facilitates the selection of transformed or transfected host cells. Suitable selectable marker genes are genes encoding proteins such as G418 and hygromycin which confer resistance to certain drugs, β-galactosidase, chloramphenicol acetyltransferase, firefly luciferase, or an immunoglobulin or portion thereof such as the Fc portion of an immunoglobulin, for example, IgG. The selectable markers may be introduced on a separate vector from the nucleic acid of interest.

Following the generation of the siRNA polynucleotide, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987, Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl- methyl-, thio- and other modified forms of adenine, cytidine, guanine, thymine, and uridine.

In one embodiment of the invention, an antisense nucleic acid sequence which is expressed by a plasmid vector is used to inhibit a T1R, a T2R, Gustducin or TrpM5 protein expression. The antisense expressing vector is used to transfect a mammalian cell or the mammal itself, thereby causing reduced endogenous expression of a T1R, a T2R, Gustducin or TrpM5.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. In one embodiment, antisense oligomers may have between about 10 to about 30 nucleotides. In one embodiment, antisense oligomers may have about 15 nucleotides. In one embodiment, antisense oligomers having 10-30 nucleotides are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

In one embodiment of the invention, a ribozyme is used to inhibit a T1R, a T2R, Gustducin or TrpM5 protein expression. Ribozymes useful for inhibiting the expression of a target molecule may be designed by incorporating target sequences into the basic ribozyme structure which are complementary, for example, to the mRNA sequence encoding a T1R, a T2R, Gustducin or TrpM5. Ribozymes targeting a T1R, a T2R, Gustducin or TrpM5, may be synthesized using commercially available reagents (Applied Biosystems, Inc., Foster City, Calif.) or they may be genetically expressed from DNA encoding them.

In one embodiment, the inhibitor of a T1R, a T2R, Gustducin or TrpM5 may comprise one or more components of a CRISPR-Cas system, where a guide RNA (gRNA) targeted to a gene encoding a T1R, a T2R, Gustducin or TrpM5, and a CRISPR-associated (Cas) peptide form a complex to induce mutations within the targeted gene. In one embodiment, the inhibitor comprises a gRNA or a nucleic acid molecule encoding a gRNA. In one embodiment, the inhibitor comprises a Cas peptide or a nucleic acid molecule encoding a Cas peptide.

Polypeptide Inhibitors

In other related aspects, the invention includes an isolated peptide inhibitor that inhibits a T1R, a T2R, Gustducin or TrpM5. For example, in one embodiment, the peptide inhibitor of the invention inhibits a T1R, a T2R, Gustducin or TrpM5 directly by binding to a T1R, a T2R, Gustducin or TrpM5 thereby preventing the normal functional activity of a T1R, a T2R, Gustducin or TrpM5. In another embodiment, the peptide inhibitor of the invention inhibits a T1R, a T2R, Gustducin or TrpM5 by competing with endogenous a T1R, a T2R, Gustducin or TrpM5. In yet another embodiment, the peptide inhibitor of the invention inhibits the activity of a T1R, a T2R, Gustducin or TrpM5 by acting as a transdominant negative mutant.

The variants of the polypeptides according to the present invention may be (i) one in which one or more of the amino acid residues are substituted with a conserved or non-conserved amino acid residue and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

Antibody Inhibitors

The invention also contemplates an inhibitor of a T1R, a T2R, Gustducin or TrpM5 comprising an antibody, or antibody fragment, specific for a T1R, a T2R, Gustducin or TrpM5. That is, the antibody can inhibit a T1R, a T2R, Gustducin or TrpM5 to provide a beneficial effect.

The antibodies may be intact monoclonal or polyclonal antibodies, and immunologically active fragments (e.g., a Fab or (Fab)₂ fragment), an antibody heavy chain, an antibody light chain, humanized antibodies, a genetically engineered single chain Fv molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Antibodies can be prepared using intact polypeptides or fragments containing an immunizing antigen of interest. The polypeptide or oligopeptide used to immunize an animal may be obtained from the translation of RNA or synthesized chemically and can be conjugated to a carrier protein, if desired. Suitable carriers that may be chemically coupled to peptides include bovine serum albumin and thyroglobulin, keyhole limpet hemocyanin. The coupled polypeptide may then be used to immunize the animal (e.g., a mouse, a rat, or a rabbit).

### Activators

In certain embodiments, the composition comprises an activator of a T1R, a T2R, Gustducin or TrpM5. In one embodiment, the activator of a T1R, a T2R, Gustducin or TrpM5 is any compound or molecule that increases the level, activity, or both of a T1R, a T2R, Gustducin or TrpM5. It will be understood by one skilled in the art, based upon the disclosure provided herein, that an increase in the level of a T1R, a T2R, Gustducin or TrpM5 encompasses the increase in a T1R, a T2R, Gustducin or TrpM5 expression, including transcription, translation, or both. The skilled artisan will also appreciate, once armed with the teachings of the present invention, that an increase in the level of a T1R, a T2R, Gustducin or TrpM5 includes an increase in a T1R, a T2R, Gustducin or TrpM5 activity (e.g., enzymatic activity, substrate binding activity, etc.). Thus, increasing the level or activity of a T1R, a T2R, Gustducin or TrpM5 includes, but is not limited to, increasing the amount of a T1R, a T2R, Gustducin or TrpM5 polypeptide, and increasing transcription, translation, or both, of a nucleic acid encoding a T1R, a T2R, Gustducin or TrpM5; and it also includes increasing any activity of a T1R, a T2R, Gustducin or TrpM5 polypeptide as well.

In one embodiment, the composition comprises an activator of T1R1, T1R2, T1R3, T2R1, T2R2, T2R3, T2R4, T2R5, T2R6, T2R7, T2R8, T2R9, T2R10, T2R11, T2R12, T2R13, T2R14, T2R15, T2R16, T2R17, T2R18, T2R19, T2R20, T2R21, T2R22, T2R24, T2R25, T2R27, T2R28, T2R29, T2R30, T2R31, T2R32, T2R33, T2R34, T2R35, T2R36, T2R37, T2R38, T2R39, T2R40, T2R41, T2R42, T2R43, T2R45, T2R46, T2R50, T2R52, T2R53, T2R54, T2R55, T2R56, T2R57, T2R58, T2R59, T2R60, T2R62P, T2R63P, T2R64P, T2R23, T2R48, T2R49, T2R26, T2R47, T2R44, T2R51, Gustudcin or TrpM5.

The increased level or activity of a T1R, a T2R, Gustducin or TrpM5 can be assessed using a wide variety of methods, including those disclosed herein, as well as methods well-known in the art or to be developed in the future. That is, the routineer would appreciate, based upon the disclosure provided herein, that increasing the level or activity of a T1R, a T2R, Gustducin or TrpM5 can be readily assessed using methods that assess the level of a nucleic acid encoding a T1R, a T2R, Gustducin or TrpM5 (e.g., mRNA), the level of a T1R, a T2R, Gustducin or TrpM5 polypeptide, and/or the level of a T1R, a T2R, Gustducin or TrpM5 activity in a biological sample obtained from a subject.

One of skill in the art will realize that in addition to activating a T1R, a T2R, Gustducin or TrpM5 directly, diminishing the amount or activity of a molecule that itself diminishes the amount or activity of a T1R, a T2R, Gustducin or TrpM5 can serve to increase the amount or activity of a T1R, a T2R, Gustducin or TrpM5. Thus, an T1R, a T2R, Gustducin or TrpM5 activator can include, but should not be construed as being limited to, a chemical compound, a protein, a peptidomemetic, an antibody, a ribozyme, and an antisense nucleic acid molecule. One of skill in the art would readily appreciate, based on the disclosure provided herein, that a T1R, a T2R, Gustducin or TrpM5 activator encompasses a chemical compound that increases the level, enzymatic activity, or substrate binding activity of a T1R, a T2R, Gustducin or TrpM5. Additionally, a T1R, a T2R, Gustducin or TrpM5 activator encompasses a chemically modified compound, and derivatives, as is well known to one of skill in the chemical arts.

The T1R, a T2R, Gustducin or TrpM5 activator compositions and methods of the invention that increase the level or activity (e.g., enzymatic activity, substrate binding activity, etc.) of a T1R, a T2R, Gustducin or TrpM5 include antibodies. The antibodies of the invention include a variety of forms of antibodies including, for example, polyclonal antibodies, monoclonal antibodies, intracellular antibodies (“intrabodies”), Fv, Fab and F(ab)2, single chain antibodies (scFv), heavy chain antibodies (such as camelid antibodies), synthetic antibodies, chimeric antibodies, and humanized antibodies. In one embodiment, the antibody of the invention is an antibody that specifically binds to a T1R, a T2R, Gustducin or TrpM5.

Further, one of skill in the art would, when equipped with this disclosure and the methods exemplified herein, appreciate that a T1R, a T2R, Gustducin or TrpM5 activator includes such activators as discovered in the future, as can be identified by well-known criteria in the art of pharmacology, such as the physiological results of activation of a T1R, a T2R, Gustducin or TrpM5 as described in detail herein and/or as known in the art. Therefore, the present invention is not limited in any way to any particular T1R, T2R, Gustducin or TrpM5 activator as exemplified or disclosed herein; rather, the invention encompasses those activators that would be understood by the routineer to be useful as are known in the art and as are discovered in the future.

Further methods of identifying and producing a T1R, a T2R, Gustducin or TrpM5 activator are well known to those of ordinary skill in the art, including, but not limited, obtaining an activator from a naturally occurring source (e.g., Streptomyces sp., Pseudomonas sp., Stylotella aurantium, etc.). Alternatively, a T1R, a T2R, Gustducin or TrpM5 activator can be synthesized chemically. Further, the routineer would appreciate, based upon the teachings provided herein, that a T1R, a T2R, Gustducin or TrpM5 activator can be obtained from a recombinant organism. Compositions and methods for chemically synthesizing T1R, T2R, Gustducin or TrpM5 activators and for obtaining them from natural sources are well known in the art and are described in the art.

One of skill in the art will appreciate that an activator can be administered as a small molecule chemical, a protein, an antibody, a nucleic acid construct encoding a protein, or combinations thereof. Numerous vectors and other compositions and methods are well known for administering a protein or a nucleic acid construct encoding a protein to cells or tissues. Therefore, the invention includes a method of administering a protein or a nucleic acid encoding a protein that is an activator of a T1R, a T2R, Gustducin or TrpM5. (Sambrook et al., 2012, Molecular Cloning: A Laboratory Manual, Cold Spring Harbor Laboratory, New York; Ausubel et al., 1997, Current Protocols in Molecular Biology, John Wiley & Sons, New York).

One of skill in the art will realize that diminishing the amount or activity of a molecule that itself diminishes the amount or activity of a T1R, a T2R, Gustducin or TrpM5 can serve to increase the amount or activity of a T1R, a T2R, Gustducin or TrpM5. Antisense oligonucleotides are DNA or RNA molecules that are complementary to some portion of a mRNA molecule. When present in a cell, antisense oligonucleotides hybridize to an existing mRNA molecule and inhibit translation into a gene product. Inhibiting the expression of a gene using an antisense oligonucleotide is well known in the art (Marcus-Sekura, 1988, Anal. Biochem. 172:289), as are methods of expressing an antisense oligonucleotide in a cell (Inoue, U.S. Pat. No. 5,190,931). The methods of the invention include the use of antisense oligonucleotide to diminish the amount of a molecule that causes a decrease in the amount or activity of a T1R, a T2R, Gustducin or TrpM5, thereby increasing the amount or activity of a T1R, a T2R, Gustducin or TrpM5. Contemplated in the present invention are antisense oligonucleotides that are synthesized and provided to the cell by way of methods well known to those of ordinary skill in the art. As an example, an antisense oligonucleotide can be synthesized to be between about 10 and about 100 nucleotides long. In one embodiment, antisense oligonucleotide can be synthesized to be between about 15 and about 50 nucleotides long. The synthesis of nucleic acid molecules is well known in the art, as is the synthesis of modified antisense oligonucleotides to improve biological activity in comparison to unmodified antisense oligonucleotides (Tullis, 1991, U.S. Pat. No. 5,023,243).

Similarly, the expression of a gene may be inhibited by the hybridization of an antisense molecule to a promoter or other regulatory element of a gene, thereby affecting the transcription of the gene. Methods for the identification of a promoter or other regulatory element that interacts with a gene of interest are well known in the art, and include such methods as the yeast two hybrid system (Bartel and Fields, eds., In: The Yeast Two Hybrid System, Oxford University Press, Cary, N.C.).

Alternatively, inhibition of a gene expressing a protein that diminishes the level or activity of a T1R, a T2R, Gustducin or TrpM5 can be accomplished through the use of a ribozyme. Using ribozymes for inhibiting gene expression is well known to those of skill in the art (see, e.g., Cech et al., 1992, J. Biol. Chem. 267:17479; Hampel et al., 1989, Biochemistry 28: 4929; Altman et al., U.S. Pat. No. 5,168,053). Ribozymes are catalytic RNA molecules with the ability to cleave other single-stranded RNA molecules. Ribozymes are known to be sequence specific, and can therefore be modified to recognize a specific nucleotide sequence (Cech, 1988, J. Amer. Med. Assn. 260:3030), allowing the selective cleavage of specific mRNA molecules. Given the nucleotide sequence of the molecule, one of ordinary skill in the art could synthesize an antisense oligonucleotide or ribozyme without undue experimentation, provided with the disclosure and references incorporated herein.

### Methods

The present invention provides methods of regulating immune cell activation and/or proliferation. In one embodiment, the invention provides a method of increasing immune cell activation by administering an inhibitor of a T1R, T2R, Gustducin or TrpM5. In one embodiment, the invention provides a method of decreasing immune cell activation by administering an activator of a T1R, T2R, Gustducin or TrpM5.

The present invention also provides methods of treating or preventing a disease or disorder associated with abnormal immune cell activation. In one embodiment, the invention provides a method for treating or preventing a disease or disorder associated with overactive immune cell activation. In one embodiment, the method of treating or preventing a disease or disorder associated with overactive immune cell activation comprise administering an activator of a T1R, a T2R, Gustducin, TrpM5, or a combination thereof. In one embodiment, diseases or disorders associated with overactive immune cell activation include, but are not limited to, inflammatory, autoimmune, and autoinflammatory diseases.

Exemplary inflammatory diseases or disorders include, but are not limited to, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, necrotizing enterocolitis, gastric and duodenal ulcers, peritonitis, organ necrosis, sepsis, endotoxic shock, cachexia, emphysema, meningitis, eczema, acne, obesity, burn, and sunburn.

In one embodiment, the diseases/disorders involving inflammation and immune cell activation are neurological and/or mental disease or disorders involving inflammation and immune cell activation. For example, in one embodiment, the neurological and/or mental disease or disorders include, but are not limited to, Parkinson's diseases, bipolar disorder, Alzheimer's disease, other forms of dementia, depression, anxiety, ADHD, autism, schizophrenia, traumatic brain injury, cerebral infarction, cerebral embolism, and migraines.

The term “autoimmune disease or disorder” refers to any disease or disorder in which the subject mounts a destructive immune response against its own tissues. The autoimmune disease or disorder can be an organ-specific disease (i.e., the immune response is specifically directed against an organ system such as the endocrine system, the hematopoietic system, the skin, the cardiopulmonary system, the gastrointestinal and liver systems, the renal system, the thyroid, the ears, the neuromuscular system, the central nervous system, gastrointestinal system, endocrine system etc.) or a systemic disease that can affect multiple organ systems.

Exemplary of autoimmune diseases include, but are not limited to Hashimoto's thyroiditis, systemic lupus erythematosus (SLE), rheumatoid arthritis (RA), polymyositis, autoimmune polyendocrinopathy syndrome type 1 (APS1)/autoimmune polyendocrinopathy-candidiasis-ectodermal dystrophy (APECED), multiple sclerosis (MS), inflammatory bowel disease (IBD), rheumatoid arthritis, juvenile rheumatoid arthritis, psoriasis, psoriatic arthritis, ankylosing spondylitis, spondyloarthritis, Sjogren's syndrome, scleroderma, lupus nephritis, polymyositis/dermatomyositis, pemphigus group diseases, bullous pemphigoid diseases, cutaneous lupus erythematosus, as type 1 diabetes, insulin dependent diabetes mellitus, ANCA-associated vasculitis (e.g., Wegener's granulomatosis, microscopic polyangiitis), Crohn's disease, Reiter's syndrome, ankylosing spondylitis, Lyme arthritis, Guillain-Barre syndrome, Graves' disease, thyroiditis, Goodpasture's syndrome, necrotizing vasculitis, lymphadenitis, ulcerative colitis, peri-arteritis nodosa, systemic sclerosis, and myasthenia gravis.

Exemplary autoinflammatory diseases or disorders include, but are not limited to, familial Mediterranean fever (FMF), neonatal onset multisystem inflammatory disease (NOMID), tumor necrosis factor (TNF) receptor-associated periodic syndrome (TRAPS), deficiency of the interleukin-1 receptor antagonist (DIRA), and Behcet's disease.

In one embodiment, the method treats or prevents a disease or disorder associated down-regulated or low immune cell activation. In one embodiment, diseases or disorders associated with down-regulated or low immune cell activation include, but are not limited to, chronic infectious diseases and cancer. In one embodiment, the low immune cell activation results from, or is an adverse effect of, a therapeutic agent. For example, in some embodiments, low immune cell activation results from, or is an adverse effect of a cancer treatment, including but not limited to, chemotherapy and radiation treatment.

In one embodiment, the disease or disorder associated with down-regulated or low immune cell activation is a chronic infectious disease. In one embodiment, the method of treating or preventing a disease or disorder associated with own-regulated or low immune cell activation comprise administering an inhibitor of a T1R, a T2R, Gustducin, TrpM5, or a combination thereof. In one embodiment, the chronic infectious disease is caused by a bacterium, virus, protozoan, helminth, or other microbial pathogen. Exemplary chronic infectious diseases include, but are not limited to, chronic viral infections including, but not limited to hepatitis (A, B, or C), herpes virus (e.g., VZV, HSV-1, HSV-6, HSV-II, CMV, and EBV), and HIV/AIDS; chronic fungal diseases including, but not limited to, Aspergillosis, Candidiasis, Coccidioidomycosis, and diseases associated with Cryptococcus and Histoplasmosis; and chronic bacterial infectious agents including, but not limited to Chlamydia pneumoniae, Listeria monocytogenes, Mycobacterium tuberculosis, Spirochaetes such as those causing Lyme disease and syphilis, influenza, malaria, schistosomaisis.

In one embodiment, the disease or disorder associated with down-regulated or low immune cell activation is cancer. The skilled artisan will understand that treating or preventing cancer in a patient includes, by way of non-limiting examples, killing and destroying a cancer cell, as well as reducing the proliferation of or cell division rate of a cancer cell. The skilled artisan will also understand that a cancer cell can be, by way of non-limiting examples, a primary cancer cell, a cancer stem cell, a metastatic cancer cell. The following are non-limiting examples of cancers that can be treated by the disclosed methods and compositions: acute lymphoblastic; acute myeloid leukemia; adrenocortical carcinoma; adrenocortical carcinoma, childhood; appendix cancer; basal cell carcinoma; bile duct cancer, extrahepatic; bladder cancer; bone cancer; osteosarcoma and malignant fibrous histiocytoma; brain stem glioma, childhood; brain tumor, adult; brain tumor, brain stem glioma, childhood; brain tumor, central nervous system atypical teratoid/rhabdoid tumor, childhood; central nervous system embryonal tumors; cerebellar astrocytoma; cerebral astrocytotna/malignant glioma; craniopharyngioma; ependymoblastoma; ependymoma; medulloblastoma; medulloepithelioma; pineal parenchymal tumors of intermediate differentiation; supratentorial primitive neuroectodermal tumors and pineoblastoma; visual pathway and hypothalamic glioma; brain and spinal cord tumors; breast cancer; bronchial tumors; burkitt lymphoma; carcinoid tumor; carcinoid tumor, gastrointestinal; central nervous system atypical teratoid/rhabdoid tumor; central nervous system embryonal tumors; central nervous system lymphoma; cerebellar astrocytoma cerebral astrocytoma/malignant glioma, childhood; cervical cancer; chordoma, childhood; chronic lymphocytic leukemia; chronic myelogenous leukemia; chronic myeloproliferative disorders; colon cancer; colorectal cancer; craniopharyngioma; cutaneous t-cell lymphoma; esophageal cancer; ewing family of tumors; extragonadal germ cell tumor; extrahepatic bile duct cancer; eye cancer, intraocular melanoma; eye cancer, retinoblastoma; gallbladder cancer; gastric (stomach) cancer; gastrointestinal carcinoid tumor; gastrointestinal stromal tumor (gist); germ cell tumor, extracranial; germ cell tumor, extragonadal; germ cell tumor, ovarian; gestational trophoblastic tumor; glioma; glioma, childhood brain stem; glioma, childhood cerebral astrocytoma; glioma, childhood visual pathway and hypothalamic; hairy cell leukemia; head and neck cancer; hepatocellular (liver) cancer; histiocytosis, langerhans cell; hodgkin lymphoma; hypopharyngeal cancer; hypothalamic and visual pathway glioma; intraocular melanoma; islet cell tumors; kidney (renal cell) cancer; langerhans cell histiocytosis; laryngeal cancer; leukemia, acute lymphoblastic; leukemia, acute myeloid; leukemia, chronic lymphocytic; leukemia, chronic myelogenous; leukemia, hairy cell; lip and oral cavity cancer; liver cancer; lung cancer, non-small cell; lung cancer, small cell; lymphoma, aids-related; lymphoma, burkitt; lymphoma, cutaneous t-cell; lymphoma, hodgkin; lymphoma, non-hodgkin; lymphoma, primary central nervous system; macroglobulinemia, waldenstrom; malignant fibrous histiocvtoma of bone and osteosarcoma; medulloblastoma; melanoma; melanoma, intraocular (eye); merkel cell carcinoma; mesothelioma; metastatic squamous neck cancer with occult primary; mouth cancer; multiple endocrine neoplasia syndrome, (childhood); multiple myeloma/plasma cell neoplasm; mycosis; fungoides; myelodysplastic syndromes; myelodysplastic/myeloproliferative diseases; myelogenous leukemia, chronic; myeloid leukemia, adult acute; myeloid leukemia, childhood acute; myeloma, multiple; myeloproliferative disorders, chronic; nasal cavity and paranasal sinus cancer; nasopharyngeal cancer; neuroblastoma; non-small cell lung cancer; oral cancer; oral cavity cancer; oropharyngeal cancer; osteosarcoma and malignant fibrous histiocytoma of bone; ovarian cancer; ovarian epithelial cancer; ovarian germ cell tumor; ovarian low malignant potential tumor; pancreatic cancer; pancreatic cancer, islet cell tumors; papillomatosis; parathyroid cancer; penile cancer; pharyngeal cancer; pheochromocytoma; paraganglioma; pineal parenchymal tumors of intermediate differentiation; pineoblastoma and supratentorial primitive neuroectodermal tumors; pituitary tumor; plasma celt neoplasm/multiple myeloma; pleuropulmonary blastoma; primary central nervous system lymphoma; prostate cancer; rectal cancer; renal cell (kidney) cancer; renal pelvis and ureter, transitional cell cancer; respiratory tract carcinoma involving the nut gene on chromosome 15; retinoblastoma; rhabdomyosarcoma; salivary gland cancer; sarcoma, ewing family of tumors; sarcoma, kaposi; sarcoma, soft tissue; sarcoma, uterine; sezary syndrome; skin cancer (nonmelanoma); skin cancer (melanoma); skin carcinoma, merkel cell; small cell lung cancer; small intestine cancer; soft tissue sarcoma; squamous cell carcinoma, squamous neck cancer with occult primary, metastatic; stomach (gastric) cancer; supratentorial primitive neuroectodermal tumors; t-cell lymphoma, cutaneous; testicular cancer; throat cancer; thymoma and thymic carcinoma; thyroid cancer; transitional cell cancer of the renal pelvis and ureter; trophoblastic tumor, gestational; urethral cancer; uterine cancer, endometrial; uterine sarcoma; vaginal cancer; vulvar cancer; waldenstrom macroglobulinemia; and wilms tumor.

It will be appreciated by one of skill in the art, when armed with the present disclosure including the methods detailed herein, that the invention is not limited to treatment of a disease or disorder associated with abnormal immune cell activation that is already established. Particularly, the disease or disorder need not have manifested to the point of detriment to the subject; indeed, the disease or disorder need not be detected in a subject before treatment is administered. That is, significant signs or symptoms of the disease or disorder do not have to occur before the present invention may provide benefit. Therefore, the present invention includes a method for preventing a disease or disorder associated with abnormal immune cell activation, in that a modulator composition, as discussed previously elsewhere herein, can be administered to a subject prior to the onset of the disease or disorder, thereby preventing the disease or disorder. The preventive methods described herein also include the treatment of a subject that is in remission for the prevention of a recurrence a disease or disorder associated with abnormal immune cell activation.

One of skill in the art, when armed with the disclosure herein, would appreciate that the prevention of a disease or disorder associated with abnormal immune cell activation, encompasses administering to a subject a modulator composition as a preventative measure against the development of, or progression of a disease or disorder associated with abnormal immune cell activation. As more fully discussed elsewhere herein, methods of modulating the level or activity of a gene, or gene product, encompass a wide plethora of techniques for modulating not only the level and activity of polypeptide gene products, but also for modulating expression of a nucleic acid, including either transcription, translation, or both.

Additionally, as disclosed elsewhere herein, one skilled in the art would understand, once armed with the teaching provided herein, that the present invention encompasses methods of treating, or preventing, a wide variety of diseases, disorders and pathologies associated with abnormal immune cell activation, where modulating the level or activity of a gene, or gene product treats or prevents the disease or disorder. Various methods for assessing whether a disease is associated with abnormal immune cell activation are known in the art. Further, the invention encompasses treatment or prevention of such diseases discovered in the future.

The invention encompasses administration of a modulator of a T1R, a T2R, Gustducin or TrpM5. To practice the methods of the invention; the skilled artisan would understand, based on the disclosure provided herein, how to formulate and administer the appropriate modulator composition to a subject. The present invention is not limited to any particular method of administration or treatment regimen.

In one embodiment, the method comprises administering to the subject in need an effective amount of a composition that modulates the expression or activity of a T1R, a T2R, Gustducin, TrpM5, or a combination thereof.

In one embodiment, the method of increasing immune cell activation and/or proliferation in a subject in need thereof comprises administering to the subject an effective amount of a composition that reduces or inhibits the expression or activity of a T1R, a T2R, Gustducin, TrpM5, or a combination thereof.

In one embodiment, the method of treating or preventing a disease or disorder associated with down-regulated or low immune cell activation and/or proliferation comprises administering to the subject an effective amount of a composition that reduces or inhibits the expression or activity of a T1R, a T2R, Gustducin, TrpM5, or a combination thereof.

In one embodiment, the method of decreasing immune cell activation and/or proliferation in a subject in need thereof comprises administering to the subject an effective amount of a composition that increases or activates the expression or activity of a T1R, a T2R, Gustducin, TrpM5, or a combination thereof.

In one embodiment, the method of treating or preventing a disease or disorder associated with overactive immune cell activation and/or proliferation comprises administering to the subject an effective amount of a composition that increases or activates the expression or activity of a T1R, a T2R, Gustducin, TrpM5, or a combination thereof.

One of skill in the art will appreciate that the modulators of the invention can be administered singly or in any combination. Further, the modulators of the invention can be administered singly or in any combination in a temporal sense, in that they may be administered concurrently, or before, and/or after each other. One of ordinary skill in the art will appreciate, based on the disclosure provided herein, that the modulator compositions of the invention can be used to prevent or to treat a disease or disorder associated with abnormal immune cell activation, and that a modulator composition can be used alone or in any combination with another modulator to achieve a therapeutic result. In various embodiments, any of the modulators of the invention described herein can be administered alone or in combination with other modulators of other molecules associated a disease or disorder associated with abnormal immune cell activation.

### Screening

In one aspect, the present invention is directed to a method for identifying compounds that regulates immune cell activation. The present invention is based, in part, described elsewhere herein, modulation of a T1R, a T2R, Gustducin or TrpM5 regulates immune cell activation. In one embodiment, the method is useful for identifying compositions that increase immune cell activation. In one embodiment, the method is useful for identifying compositions that decreases immune cell activation.

In one embodiment, the method is useful for identifying compositions that treat a disease or disorder is associated with abnormal immune cell activation. In one embodiment, the method is useful for identifying compositions that treat a disease or disorder is associated with overactive immune cell activation. In some embodiments, the method is useful for identifying compositions that treat a disease or disorder is associated with down-regulated or low immune cell activation.

In one embodiment, the method comprises contact a test composition with a sample; detecting the expression, activity, or both of T1Rs, T2Rs, Gustducin, TrpM5, or a combination thereof; and identifying a modulator of the T1R, T2R, Gustducin or TrpM5, wherein the modulator regulates immune cell activation.

In one embodiment, the modulator of the T1R, T2R, Gustducin or TrpM5 is an inhibitor of T1R, T2R, Gustducin or TrpM5. In one embodiment, the inhibitor of T1R, T2R, Gustducin or TrpM5 increases immune cell activation. In one embodiment, the inhibitor of T1R, T2R, Gustducin or TrpM5 is useful for treating a disease or disorder is associated with down-regulated or low immune cell activation.

In one embodiment, the modulator of the T1R, T2R, Gustducin or TrpM5 is an activator of T1R, T2R, Gustducin or TrpM5. In one embodiment, the activator of the T1R, T2R, Gustducin or TrpM5 decreases immune cell activation. In one embodiment, the inhibitor of the T1R, T2R, Gustducin or TrpM5 is useful for treating a disease or disorder is associated with overactive immune cell activation.

In one embodiment, the screening method of the present invention is an in vitro assay. For example, in one embodiment, the method comprises contacting at least one recombinant T1R, T2R, Gustducin, or TrpM5 with a test compound and detecting the activity or expression of T1R, T2R, Gustducin, TrpM5.

In one embodiment, the screening method of the present invention is a cell-based assay. For example, in one embodiment the method comprises contacting a cell with a test composition and detecting the expression, activity, or both of T1Rs, T2Rs, Gustducin, TrpM5, or a combination thereof, using the methods described herein. The cell may be cultured with the test composition for a defined time period prior to determining activity or expression. For example, in certain embodiments, the cell may be cultured with the test composition for about 1 second, 5 seconds, 10 seconds, 30 seconds, 1 minute, 2 minutes, 5 minutes, 10 minutes, 30 minutes, 1 hour, 2 hours, 6 hours, 12 hours, 24 hours, 3 days, 7 days, 2 weeks, 1 month, 3 months, or longer. It can be determined if the test composition alters the expression or activity of T1Rs, T2Rs, Gustducin, or TrpM5 as compared to a similar cell which is not cultured with the test composition. Aside from the particular composition or condition being screened, the cell may be cultured using any standard culture conditions or cell culture media known in the art.

Any suitable cell may be used for the cell-based assay including, but not limited to, prokaryotic cells, eukaryotic cells, and mammalian cells. In one embodiment, the cell is a cell that expresses at least one of a T1R, a T2R, Gustducin, and TrpM5. In one embodiment, the cell is modified to express at least one recombinant T1R, T2R, Gustducin or TrpM5. In one embodiment, the cell-based assay comprises one or more cells derived from a cell line including, but not limited to, HEK 293T, CHO, BHK, VERO, HeLa, COS, MDCK, NSO and W138. In one embodiment, the cell based assay comprises one or more primary cells isolated from a subject (e.g. a mammal). For example, in one embodiment, the assay comprises the use of a immune cell isolated from a subject. In one embodiment, the cell based screen comprises an in vivo screening assay, wherein the recombinant protein is introduced into one or more cells in animal. In some embodiments, a cell based assay is used as a secondary screen on test compounds identified as modulators of a T1R, a T2R, Gustducin, or TrpM5 expression or activity in an in vitro screening assay.

In certain embodiments, the method is a high throughput method, where a plurality of test compositions or conditions are screened. For example, in certain embodiments, a library of compositions is screened, where each composition of the library is individually contacted to a sample in order to identify which compositions modulate or do not modulate the activity, expression or both of a T1R, a T2R, Gustducin or TrpM5.

The test compounds can be obtained using any of the numerous approaches in combinatorial-library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam et al., 1997, Anticancer Drug Des. 12:45).

Examples of methods for the synthesis of molecular libraries can be found in the art, for example, in: DeWitt et al., 1993, Proc. Natl. Acad. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233.

Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria (Ladner U.S. Pat. No. 5,223,409), spores (Ladner U.S. Pat. No. '409), plasmids (Cull et al., 1992, Proc. Natl. Acad. Sci. USA 89:1865-1869) or on phage (Scott and Smith, 1990, Science 249:386-390; Devlin, 1990, Science 249:404-406; Cwirla et al., 1990, Proc. Natl. Acad. Sci. USA 87:6378-6382; Felici, 1991, J. Mol. Biol. 222:301-310; and Ladner supra).

In situations where “high-throughput” modalities are used, it is typical that new chemical entities with useful properties are generated by identifying a chemical compound (called a “lead compound”) with some desirable property or activity, creating variants of the lead compound, and evaluating the property and activity of those variant compounds. The current trend is to shorten the time scale for all aspects of drug discovery.

In one embodiment, high throughput screening methods involve providing a library containing a large number of compounds (candidate compounds) potentially having the desired activity. Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

### Pharmaceutical Compositions and Formulations

The invention also encompasses the use of pharmaceutical compositions of the invention or salts thereof to practice the methods of the invention. Such a pharmaceutical composition may consist of at least one modulator composition of the invention or a salt thereof in a form suitable for administration to a subject, or the pharmaceutical composition may comprise at least one modulator composition of the invention or a salt thereof, and one or more pharmaceutically acceptable carriers, one or more additional ingredients, or some combination of these. The compound or conjugate of the invention may be present in the pharmaceutical composition in the form of a physiologically acceptable salt, such as in combination with a physiologically acceptable cation or anion, as is well known in the art.

In an embodiment, the pharmaceutical compositions useful for practicing the methods of the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 500 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for oral, rectal, vaginal, parenteral, topical, pulmonary, intranasal, buccal, ophthalmic, or another route of administration. A composition useful within the methods of the invention may be directly administered to the skin, vagina or any other tissue of a mammal. Other contemplated formulations include liposomal preparations, resealed erythrocytes containing the active ingredient, and immunologically-based formulations. The route(s) of administration will be readily apparent to the skilled artisan and will depend upon any number of factors including the type and severity of the disease being treated, the type and age of the veterinary or human subject being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist may design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In one embodiment, the compositions of the invention are formulated using one or more pharmaceutically acceptable excipients or carriers. In one embodiment, the pharmaceutical compositions of the invention comprise a therapeutically effective amount of a compound or conjugate of the invention and a pharmaceutically acceptable carrier. Pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In one embodiment isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin. In one embodiment, the pharmaceutically acceptable carrier is not DMSO alone.

Formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. An exemplary preservative is a combination of about 0.5% to 2.0% benzyl alcohol and 0.05% to 0.5% sorbic acid.

In one embodiment, the composition includes an anti-oxidant and a chelating agent that inhibits the degradation of the compound. Exemplary antioxidants for some compounds are BHT, BHA, alpha-tocopherol and ascorbic acid in the range of about 0.01% to 0.3%. In one embodiment, the BHT is in the range of 0.03% to 0.1% by weight by total weight of the composition. In one embodiment, the chelating agent is present in an amount of from 0.01% to 0.5% by weight by total weight of the composition. Exemplary chelating agents include edetate salts (e.g. disodium edetate) and citric acid in the weight range of about 0.01% to 0.20%. In one embodiment, chelating agents may be in the range of 0.02% to 0.10% by weight by total weight of the composition. The chelating agent is useful for chelating metal ions in the composition that may be detrimental to the shelf life of the formulation. While BHT and disodium edetate are the exemplary antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

The regimen of administration may affect what constitutes an effective amount. The therapeutic formulations may be administered to the subject either prior to or after a diagnosis of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, such a mammal, including a human, may be carried out using known procedures, at dosages and for periods of time effective to prevent or treat disease. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

The compound may be administered to a subject as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In certain embodiments, the composition of the present invention provides for a controlled release of a therapeutic agent, such as a modulator of a T1R, a T2R, Gustducin, or TrpM5. In certain instances, controlled- or sustained-release formulations of a pharmaceutical composition of the invention may be made using conventional technology, using for example proteins equipped with pH sensitive domains or protease-cleavable fragments. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, micro-particles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for oral administration, such as tablets, capsules, gel-caps, lozenges, and caplets, which are adapted for controlled-release are encompassed by the present invention.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased subject compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In certain embodiments, the controlled-release formulation of the composition described herein allows for release of a therapeutic agent precisely when the agent is most needed. In another embodiment, the controlled-release formulation of the composition described herein allows for release of a therapeutic agent precisely in conditions in which the therapeutic agent is most active. In order to maintain this constant level of drug in the body, the drug must be released from the dosage form at a rate that will replace the amount of drug being metabolized and excreted from the body.

In certain embodiment, the composition provides for an environment-dependent release, when and where the therapeutic agent is triggered for release. For example, in certain embodiments the composition invention releases at least one therapeutic agent when and where the at least one therapeutic agent is needed. The triggering of release may be accomplished by a variety of factors within the microenvironment of the treatment or prevention site, including, but not limited to, temperature, pH, the presence or activity of a specific molecule or biomolecule, and the like.

Controlled-release of an active ingredient can be stimulated by various inducers, for example pH, temperature, enzymes, water or other physiological conditions or compounds. The term “controlled-release component” in the context of the present invention is defined herein as a compound or compounds, including, but not limited to, polymers, polymer matrices, gels, permeable membranes, liposomes, or microspheres or a combination thereof that facilitates the controlled-release of the active ingredient.

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release that is longer that the same amount of agent administered in bolus form.

For sustained release, the compounds may be formulated with a suitable polymer or hydrophobic material that provides sustained release properties to the compounds. As such, the compounds for use the method of the invention may be administered in the form of microparticles, for example, by injection or in the form of wafers or discs by implantation.

In one embodiment of the invention, the compounds of the invention are administered to a subject, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

In one embodiment, the compositions of the invention are administered to the subject in dosages that range from one to five times per day or more. In another embodiment, the compositions of the invention are administered to the subject in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Compounds of the invention for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 3050 mg to about 6,000 mg, about 500 mg to about 5,000 mg, about 750 mg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., a drug used for treating the same or another disease as that treated by the compositions of the invention) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a compound or conjugate of the invention, alone or in combination with a second pharmaceutical agent; and instructions for using the compound or conjugate to treat, prevent, or reduce one or more symptoms of a disease in a subject.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating or preventing a disease in a subject, or delivering an imaging or diagnostic agent to a subject.

Routes of administration of any of the compositions of the invention include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out certain embodiments of the present invention, and are not to be construed as limiting in any way the remainder of the disclosure.

### Example 1: Regulation of Immune Cell Activation and Proliferation Via Taste Receptor-, Gustducin-, and TrpM5-Mediated Pathways

The data presented herein demonstrates novel strategies for regulating immune cell activation and proliferation in vivo and in vitro via taste receptor-, Gustducin-, and TrpM5-mediated pathways. A novel pathway is described for direct regulation of immune cell activation and proliferation that involves taste receptors, Gustducin, and TrpM5 expressed in immune cells.

### Expression of Taste Receptors, Gustducin, and TrpM5 in Immune Organs and Cells

G-protein-coupled taste receptors, T1Rs and T2Rs, and their downstream signaling molecules, such as Gustducin and TrpM5, were discovered from taste bud cells in the oral cavity. However, recent evidence shows that these molecules are expressed in multiple extra-oral tissues and play various biological functions.

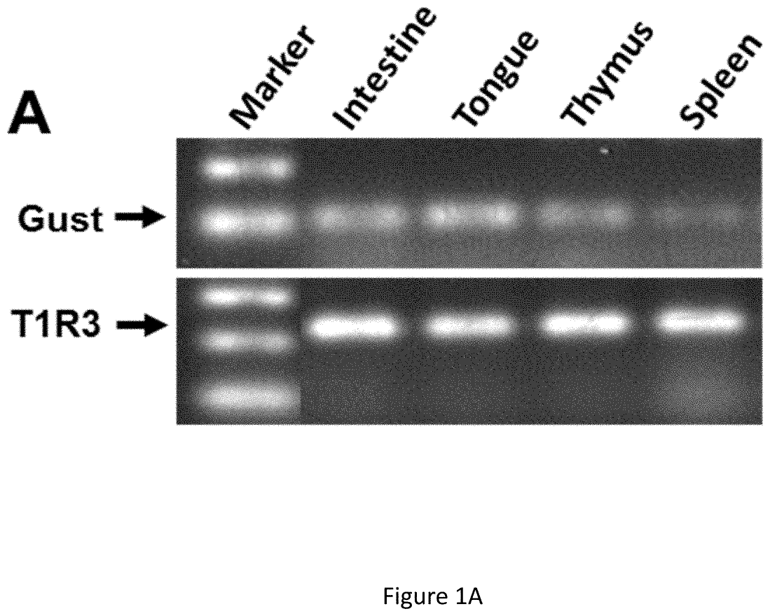

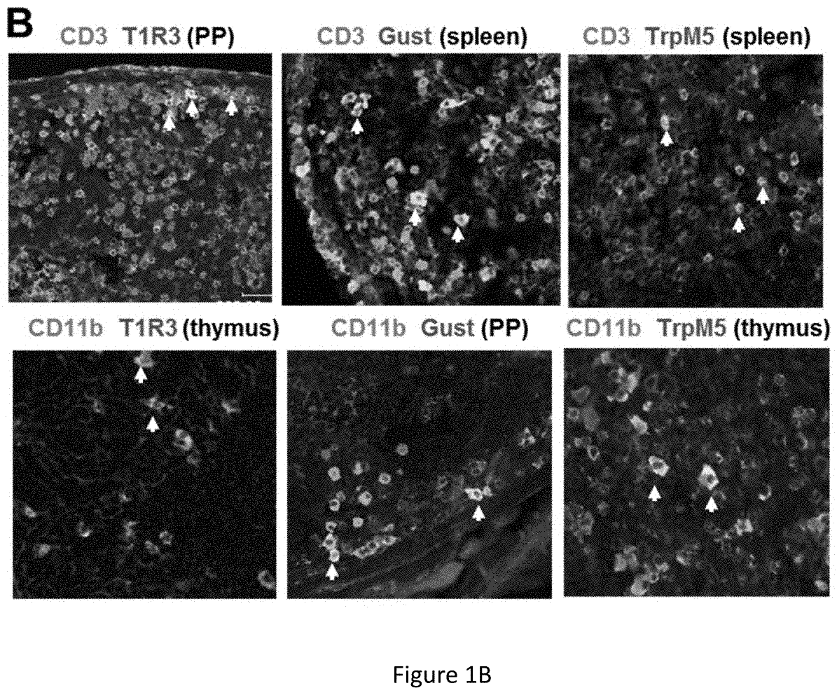

To determine if taste receptors and their downstream signaling molecules are involved in direct regulation of immune cell function, the expression of these molecules in immune organs and cultured immune cells was studied. FIG. 1A shows PCR detection of T1R3 and Gustducin expression in the intestine, tongue, thymus, and spleen, the latter two are immune organs containing primarily immune cells. The results show that both gustducin and T1R3 are expressed in the thymus and spleen. To study what types of cells in immune organs and structures express taste receptors and their signaling proteins, immunostaining experiments were performed using antibodies against T1R3, Gustducin, TrpM5, and various immune cell type markers. The results show that T cells, macrophages, neutrophils, and dendritic cells all express T1R3, Gustducin, and TrpM5 (FIG. 1B).

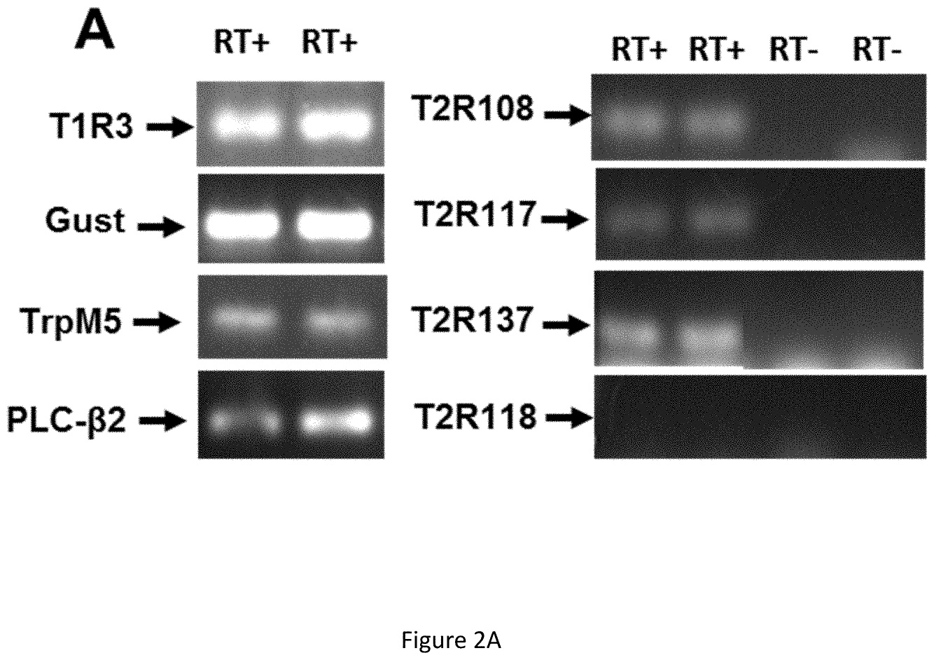

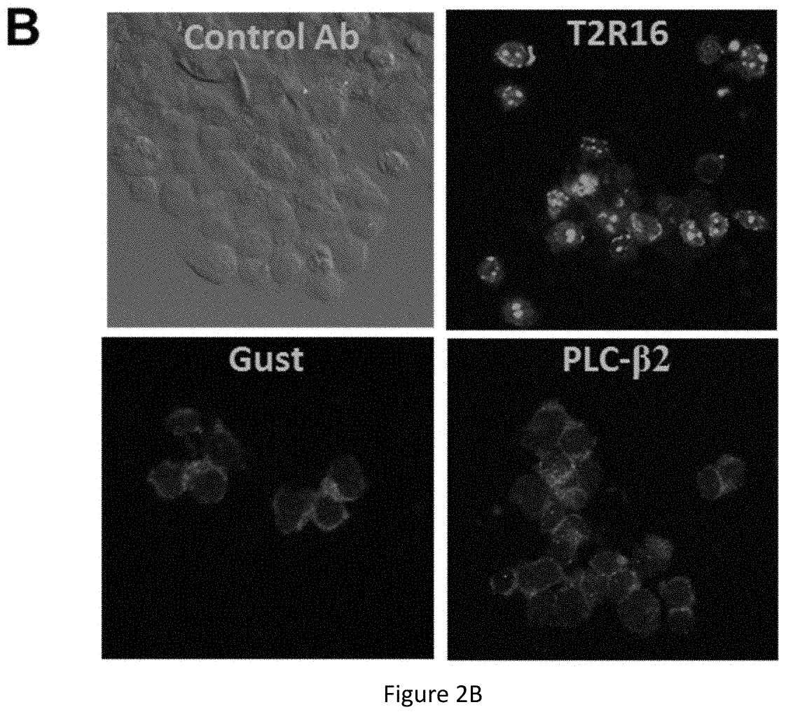

Cultured cell lines were used to verify the expression of taste receptors, Gustducin, and TrpM5 in immune cells. FIG. 2 shows taste receptors (including T1Rs and some T2Rs), Gustducin, TrpM5, and PLC-β2 expression in EL-4 cells (a mouse T cell line). Taste receptors, Gustducin, TrpM5, and PLC-β2 expression was also detected in RAW264.7 cells (a mouse monocyte/macrophage cell line) (data not shown).

Together, these results show that taste receptors (including T1Rs and a subset of T2Rs) and their signaling molecules, including gustducin, TrpM5, and PLC-β2, are expressed in immune organs (spleen and thymus) and structures (Peyer's patch), and particularly in T cells and monocytes/macrophages.

T1R3, Gustducin, and TrpM5 are Involved in Immune Cell Activation and Proliferation

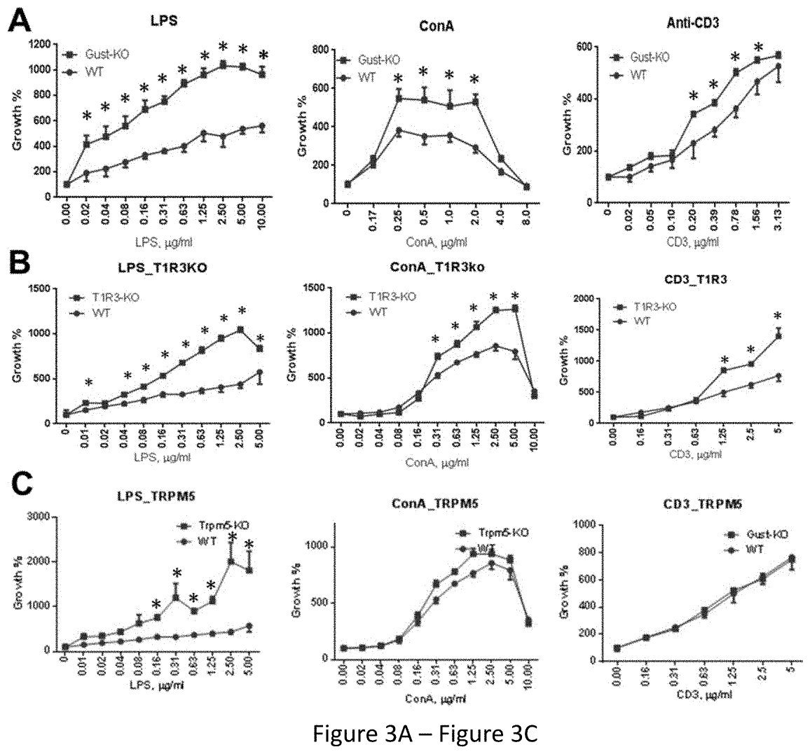

To investigate the functional involvement of the taste-like chemosensory pathway in immune cells, in vitro immune cell activation assays were carried out. For these assays, spleen cells (splenocytes, including T cells, B cells, dendritic cells, and monocytes, but not red blood cells which were depleted from the cell pool) were isolated from gustducin knockout, T1R3 knockout, TrpM5 knockout, and wild-type control mice. Splenocytes from these mice were then treated with three immune cell activators, lipopolysaccharide (LPS, a broad immune cell activator), Concanavalin A (ConA, an activator of T and B cells), and an antibody specific to CD3 (a specific activator of T cells). Activation of immune cells was monitored by measuring cell proliferation (a commonly used method for measuring immune cell activation). As shown in FIG. 3, lacking functional Gustducin (FIG. 3A), T1R3 (FIG. 3B), or TrpM5 (FIG. 3C) results in profound hyper-activation of immune cells, especially under LPS-induced immune cell activation (left panels). Gustducin and T1R3 knockout splenocytes also showed hyper-activation when induced with ConA (middle panels) and anti-CD3 (right panels). These results strongly suggest that the taste-like chemosensory pathway in immune cells plays important roles in immune cell activation and proliferation.

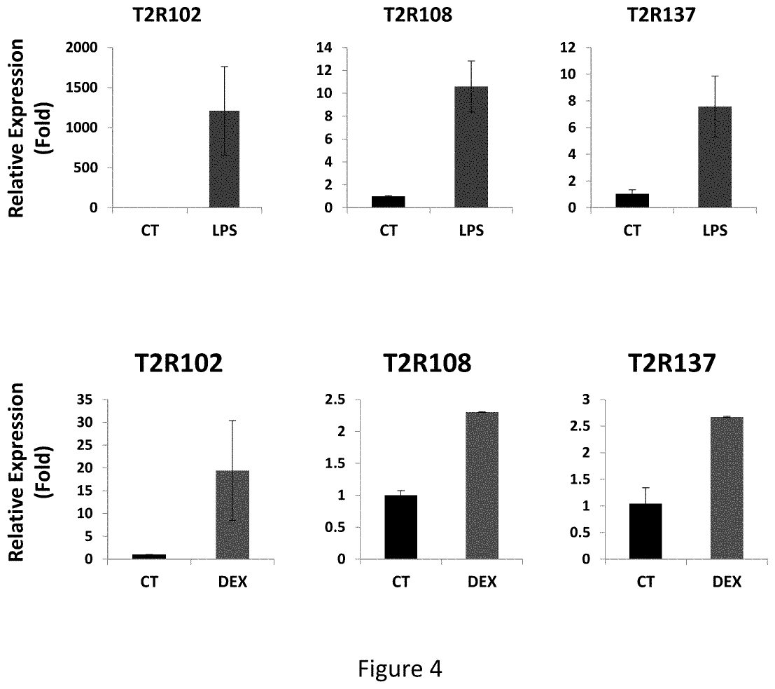

The mouse monocyte/macrophage Raw264.7 cell line was also used to study T2Rs. Raw264.7 cells were treated with lipopolysaccharide (LPS) and dexamethasone (DEX). The expression levels of some bitter receptors (T2Rs) can be strongly induced by LPS and moderately induced by DEX (FIG. 4). These results suggest that the functions of T2Rs can be strengthened by the Toll-like receptor and the glucocorticoid receptor pathways.

These results indicate that by manipulating the activities of taste receptors, Gustducin and TrpM5 in immune cells, one can regulate the activation and proliferation of immune cells. This idea can be applied to treat various diseases. For instance, agonists of T1Rs and T2Rs, as well as activators of Gustducin and TrpM5, can be used to inhibit immune cell activation and proliferation in diseases in which immune cells are overly activated, such as in inflammatory, autoimmune, and autoinflammatory diseases. On the other hand, antagonists of T1Rs and T2Rs, as well as inhibitors of Gustducin and TrpM5, can be used to stimulate immune cell activation and proliferation either in vivo or in vitro (splenocyte, T cells, macrophages, or other immune cell cultures) to treat diseases in which immune cell function is inhibited, such as in cancer environment and in chronic infectious diseases.

### Example 2: Aggravated Gut Inflammation in Mice Lacking the Taste Signaling Protein α-Gustducin

The results presented herein show that α-gustducin knockout mice with DSS-induced colitis have aggravated weight loss, diarrhea, intestinal bleeding, and inflammation over the experimental period compared to wild-type mice, concurrent with augmented immune cell infiltration and increased expression of TNF and IFN-γ but decreased expression of IL-13 and IL-5 in the colon. These results suggest that the taste receptor signaling pathway may play critical roles in regulating gut immune balance and inflammation. Further, these results show that loss of function of α-gustducin, a principal component for taste GPCR signaling, leads to aggravated colitis in mice, with excessive weight loss, tissue damage, and inflammation compared to wild-type controls. Cytokine profiling shows that colons from mice lacking functional α-gustducin express lower levels of the type II cytokines interleukin-13 (IL-13) and IL-5 but higher levels of tumor necrosis factor (TNF) and interferon-γ (IFN-γ) than do colons from wild-type controls. These results suggest that the taste-signaling protein α-gustducin is important for controlling immune balance, inflammation, and tissue integrity in gut.

The materials and methods are now described

Animals

α-Gustducin-knockout mice were described previously (Wong et al., 1996, Nature 381:796-800) and have since been backcrossed with C57BL/6 for at least ten generations. Knockout (α-gustducing−/−) and wild-type control (α-gustducin+/+) mice were kept in a specific pathogen free (SPF) animal facility. For each experiment, mice from several different cages and breeder pairs, including both male and female, were used. Age, gender, and body weight were matched between wild-type and knockout mice.

Colitis Induction and Disease Evaluation

Experimental colitis was induced by giving mice 3% DSS in drinking water, ad libitum, for 7 days. The intake of DSS solution per animal was recorded. Baseline body weight was measured before DSS administration. During DSS administration, mouse body weight was measured daily. Mice were also observed daily for diarrhea and rectal bleeding. The following modified scoring system, was used for measuring disease index (Chassaing et al., 2014, Curr Protoc Immunol 104): 0 for normal stool with no blood, 1 for soft stool with no blood, 2 for soft stool with little blood, 3 for very soft stool with modest bleeding, and 4 for watery stool with significant bleeding. By the end of the 7-day period, mice were euthanized, and the weights of the spleen and the length of the colon were measured. Gut tissues were collected for histological and gene expression analysis. Three independent experiments have been conducted.

Histological Analysis

For evaluation of histopathology, untreated and DSS-treated mice were sacrificed on day 7. Proximal, middle, and distal sections of the colon were collected for histology. Colon sections were stained with H&E and evaluated for epithelial damage. Histological scoring was performed on H&E-stained colon tissue sections. Each tissue section was scored on the degree of epithelial damage and inflammation in the mucosa, submucosa, and muscularis and serosa regions. Individual scores were assigned as follows: 0 for no tissue damage and inflammation, 1 for focal damage and inflammation, 2 for patchy tissue damage and inflammation, and 3 for diffuse tissue damage and inflammation. Four scores per mouse (one each for proximal, middle, and distal parts of colon, and one for cecum) were added for the total score of the individual animal. The average scores for each group were calculated.

Immunohistochemistry

Specific antibodies against leukocytes (CD45), T cells (CD3), B cells (B220), macrophages(CD11b), and neutrophils (Ly6G) were used to label different types of immune cells in the gut tissues as described previously (Feng et al., 2009, Brain Behav Immun. 23:760-66). The populations of identified immune cells in both the epithelium and the lamina propria underneath the epithelium were quantitatively measured using Image-Pro Plus software. The infiltrated cells in the tissues are expressed as the ratio of the stained area to the total area of tissue region measured.

Gene Expression Analysis

For gene expression analysis, colon tissues from untreated and DSS-treated wild-type and knockout mice were collected. Total RNA was isolated, treated with DNase I to remove genomic DNA contamination, and then reverse transcribed into cDNA. Real-time quantitative PCR (qPCR) was performed to measure gene expression levels as described previously (Feng et al., 2014 J Neurosci 24:2689-701). qPCR primers are listed in Table 1. Relative quantification of gene expression was determined using the 2^{-ΔΔCt} method.

TABLE 1
qPCR primers

| | Accession | | | Product |
|---|---|---|---|---|
| Gene | No. | Orientation | Sequence 5′ to 3′ | Size (bp) |
| B-actin | NM_007393 | Forward | GATTACTGCTCTGGCTCCTA (SEQ ID NO: 4) | 142 |
| | | Reverse | ATCGTACTCCTGCTTGCTGA (SEQ ID NO: 5) | |
| TNF | NM_013693 | Forward | CTTCTCATTCCTGCTTGTGG (SEQ ID NO: 6) | 140 |
| | | Reverse | ATCTGAGTGTGAGGGTCTGG (SEQ ID NO: 7) | |
| IFN-γ | NM_008337 | Forward | AGCAACAGCAAGGCGAAAA (SEQ ID NO: 8) | 71 |
| | | Reverse | CTGGACCTGTGGGTTGTTGA (SEQ ID NO: 9) | |
| IL-5 | NM_010558 | Forward | AGCAATGAGACGATGAGGCTT (SEQ ID NO: 10) | 124 |
| | | Reverse | GCATTTCCACAGTACCCCCA (SEQ ID NO: 11) | |
| IL-13 | NM_008355 | Forward | ACAAGACCAGACTCCCCTGT (SEQ ID NO: 12) | 128 |
| | | Reverse | TCTGGGTCCTGTAGATGGCA (SEQ ID NO: 13) | |
| IL-10 | NM_010548 | Forward | AAGGCAGTGGAGCAGGTGAA (SEQ ID NO: 14) | 159 |
| | | Reverse | CCAGCAGACTCAATACACAC (SEQ ID NO: 15) | |
| TGF-β1 | NM_011577 | Forward | AGAGAAGAACTGCTGTGTGC (SEQ ID NO: 16) | 176 |
| | | Reverse | GGGTTGTGTTGGTTGTAGAG (SEQ ID NO: 17) | |

Data Analysis

Weight loss and disease index data were first compiled using Microsoft Excel. For statistical analyses, ANOVA with post hoc t-tests were performed using Statistica. Two-tailed Student's t-tests were performed for gene expression analyses and immune cell infiltration experiments. Ap-value<0.05 was considered statistically significant.

The results are now described.

Mice Null for α-Gustducin are Highly Susceptible to DSS-Induced Colitis

To investigate whether the taste-like chemosensory pathways in the gut are involved in regulating colitogenic responses, α-gustducin-knockout mice were tested in the well-established DSS-induced colitis model. Knockout and wild-type control mice were given 3% DSS for 7 days to induce colitis. No significant differences were observed in DSS intake between wild-type and knockout mice (data not shown).

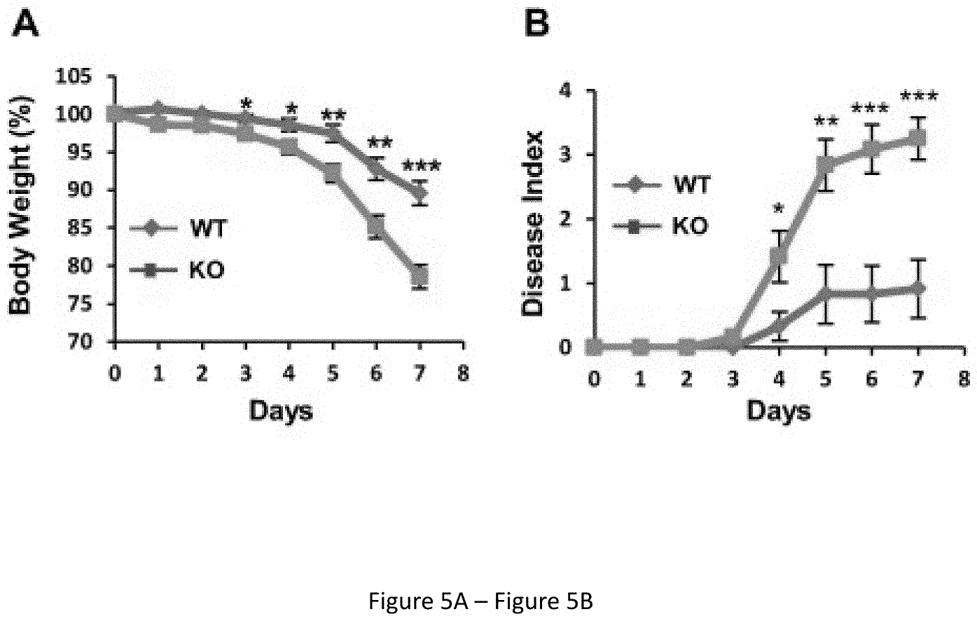

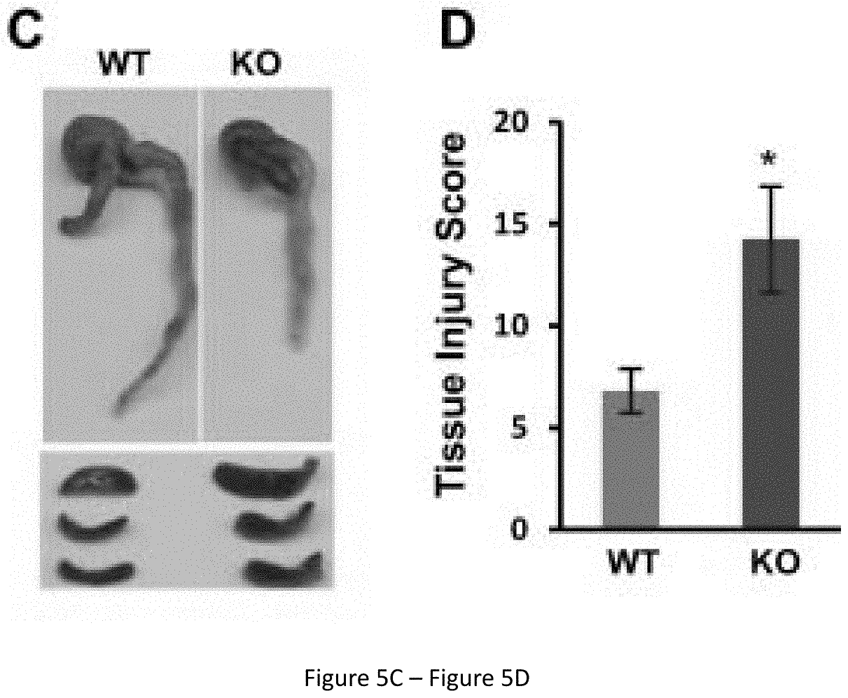

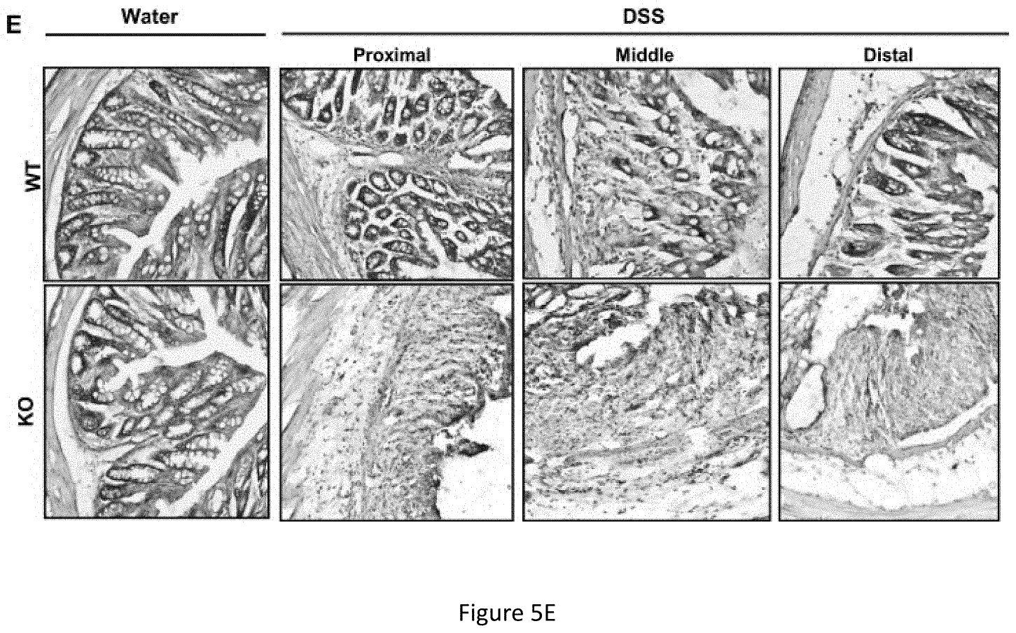

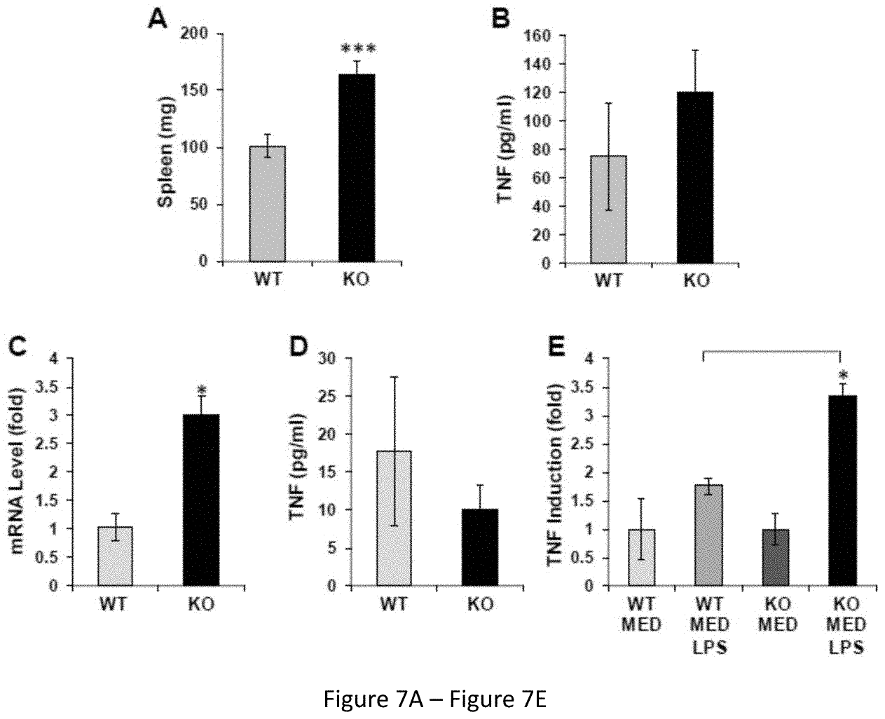

The manifestations of DSS-induced colitis were examined. FIG. 5 shows that α-gustducin-null mice developed much more severe colitis than did wild-type mice. Mice null for α-gustducin had accelerated and severe weight loss compared to wild-type controls during DSS administration (FIG. 5A). Their disease index, which was based on the severity of diarrhea and rectal bleeding, was significantly higher than that of wild-type mice (FIG. 5B). The colon of DSS-treated α-gustducin-null mice was much shorter than that of wild-type mice (FIG. 5C, upper panel), another indication of more severe colitis in these knockout mice. Mice null for α-gustducin also had significantly enlarged spleen than did wild-type mice (FIG. 5C, lower panel and FIG. 7A), suggesting higher inflammatory responses.

Histopathological changes were also examined in the colon for tissue damage related to colitis. Without DSS administration, the colon of α-gustducin-knockout mice displayed normal histological structure that was indistinguishable from that of wild-type mice (FIG. 5E, left panels). DSS administration resulted in more severe tissue damage in the colon of knockout mice than in the colon of wild-type mice (FIG. 5E, DSS). Mice null for α-gustducin had much more intensive and extensive crypt loss, inflammatory cell infiltration, epithelial-erosion, and ulceration than did wild-type mice in the proximal, middle, and distal regions of the colon. Histological scoring showed significantly higher tissue injury scores in DSS-treated α-gustducin-null mice than in wild-type mice (FIG. 5D). These results indicate that α-gustducin is critical for protecting mice against DSS-induced intestinal tissue damage.

α-Gustducin-Knockout Mice Display Increased Colonic Inflammatory Responses in the DSS Model

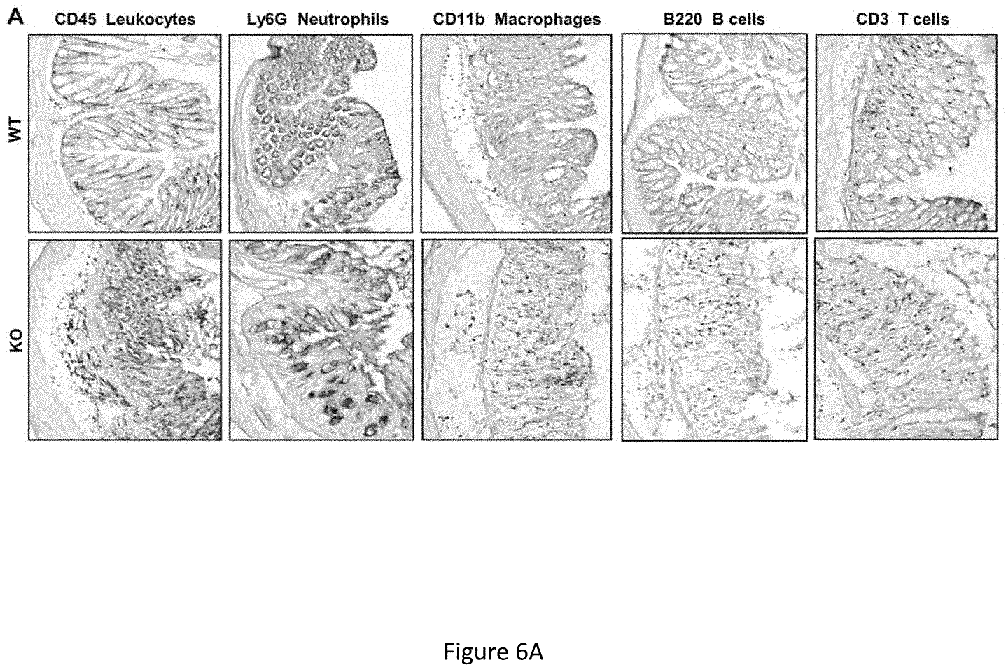

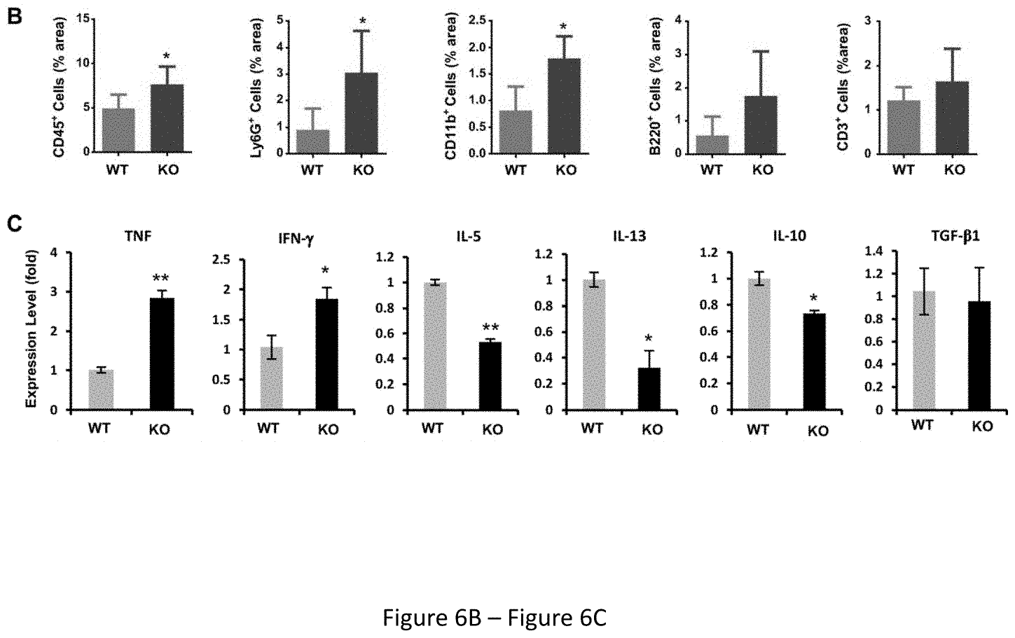

The innate and adaptive immune systems in the gut mucosa protect the intestine from potential infectious agents and play important roles in maintaining tissue homeostasis. However, excessive immune activation contributes to IBD. In DSS-induced colitis, infiltration of various inflammatory cells, such as neutrophils and macrophages, is a common feature of colitis and an important parameter for evaluating the severity of the disease (Chassaing et al., 2014, Curr Protoc Immunol 104). To assess whether α-gustducin affects immune cell infiltration in DSS-induced colitis and to determine which types of immune cells might be affected immunostaining experiments were performed using antibodies against various immune cell markers, including the pan-leukocyte marker CD45, neutrophil marker LyG6, macrophage marker CD11b, B cell marker B220, and T cell marker CD3. A marked increase in immune cell infiltration was observed on colon sections of DSS-treated α-gustducin-knockout mice compared to wild-type mice (FIG. 6A). Quantitative analyses revealed significant increases in the number of total leukocytes, neutrophils, and macrophages in colons of α-gustducin-knockout mice compared to wild-type mice (FIG. 6B). The numbers of T and B cells also appeared to be higher in colons of the knockout mice than in wild-type mice, although the differences did not reach statistical significance (FIG. 6B). These results are consistent with the aggravated colitis in DSS-treated α-gustducin-null mice and suggest that lacking the taste-signaling protein leads to excessive inflammation caused by colitogenic agents.

Inflammatory cytokines contribute to the pathogenesis of IBD. Increased levels of inflammatory cytokines, particularly TNF, in the gut mucosa are observed in patients with active IBD (Reimund et al., 1996, J Clin Immunol 16:144-50). Anti-TNF therapies have shown efficacy in treating Crohn's disease and ulcerative colitis (Nielsen and Ainsworth, 2013, NEJM 369:754-62). To examine whether the aggravated colitis observed in α-gustducin-knockout mice is associated with higher levels of inflammatory cytokines, quantitative RT-PCR experiments were performed to evaluate cytokine levels in colons of DSS-treated mice. The results show that the levels of TNF and IFN-γ were significantly higher in colons of DSS-treated knockout mice than in wild-type mice (FIG. 6C). The mean blood level of TNF was also higher in DSS-treated α-gustducin knockout mice than that in wild-type mice, although the difference did not reach statistically significant level (FIG. 7B). In contrast, the level of the anti-inflammatory cytokine IL-10 was lower in colons of α-gustducin-null mice than that in wild-type mice. The levels of TGF-01, another cytokine with anti-inflammatory activities, did not significantly differ between wild-type and knockout mice (FIG. 6C). Interestingly, the mRNA level of TNF in non-stimulated colon of α-gustducin knockout mice was also significantly higher than that of wild-type mice (FIG. 7C). Lipopolysaccharide stimulated higher level of TNF induction in cultured colon explants from α-gustducin knockout mice than that from wild-type mice, even though TNF secretion in non-stimulated colon explants of the knockout mice did not differ from those of wild-type mice (FIG. 7D and FIG. 7E). These results suggest that lacking α-gustducin may potentiate the induction of TNF by increasing the basal level of TNF mRNA expression. Together, these results show that mice lacking functional α-gustducin developed higher levels of inflammatory responses in DSS-induced colitis than did wild-type mice, consistent with the observed higher levels of immune cell infiltration.

Imbalance of immune regulation is another mechanism contributing to IBD. Imbalance of T-helper type 1 (Th1) to Th2 cells has been proposed as a model to explain the increased prevalence of allergic, inflammatory, and autoimmune diseases in developed countries (Wills-Karp et al., 2001, Nat Rev Immunol 1:69-75). Decreased type II immunity may favor type I-driven inflammatory responses. Considering the recent findings on the roles of the taste-like chemosensory pathways in establishing type II immune responses to gut parasites (Howitt et al., 2016, Science 341:1329-33), it was investigated whether lacking functional taste-signaling proteins would reduce levels of type II cytokines in the DSS-induced colitis model. The expression levels of the type II cytokines IL-5 and IL-13 were measured in the colons of DSS-treated mice. As shown in FIG. 6C, the expression levels of IL-5 and IL-13 were significantly lower in the colons of α-gustducin-knockout mice than in wild-type mice. These results suggest that deficiency in the taste signaling pathway may lead to an imbalance of type I and II responses in the DSS colitis model, which may contribute to the aggravated inflammation and disease manifestation observed in α-gustducin-knockout mice.

In this study, the role of a key taste-signaling protein, α-gustducin, in DSS-induced experimental colitis was examined. These results clearly show that mice lacking functional α-gustducin are more susceptible to DSS-induced colitis with more severe tissue damage and excessive inflammatory responses. The higher levels of TNF and IFN-γ and lower levels of IL-10, IL-5, and IL13 in the colon of α-gustducin-knockout mice suggest that lacking functional taste-signaling skews immune responses more toward the inflammatory, type I immune response than the anti-inflammatory, type II response. This imbalanced immune response could be responsible for the observed severe colitis phenotype in α-gustducin-knockout mice. The molecular mechanisms for the skewed immune responses warrant further investigation. It was previously shown that taste bud cells can produce multiple cytokines, including both inflammatory and anti-inflammatory cytokines (Feng et al., 2014, J Neurosci 24:2689-701). Tuft cells in the gut share many similarities with taste bud chemosensory cells. In response to parasites, tuft cells release IL-25 through a taste chemosensory pathway-dependent mechanism (Howitt et al., 2016, Science 341:1329-33). How the taste or taste-like chemosensory pathways in the gut contribute to type II immunity still remains unclear. In this study we used conventional (global) knockout of α-gustducin, which cannot determine which tissue(s) or cell type(s) play major roles in the immune regulatory mechanism. Future studies using tissue- or cell-type-specific knockout of α-gustducin will provide further mechanistic insights. In addition, knockout of α-gustducin is known to affect the secretion of various gut hormones, such as glucagon-like peptide-1 and glucose-dependent insulinotropic peptide (Jang et al., 2007, PNAS 104:15069-74; Kokrashvili et al., 2009, Am J Clin Nutr 90:822S-825S). Whether reduced secretion of these gut hormones also contributes to the aggravated inflammation in DSS-treated α-gustducin knockout mice remains to be determined.

T1Rs and T2Rs are the most intensively studied and best-characterized taste GPCRs. However, other receptors, such as the proposed lipid or fat taste receptors GPR120 and GPR40, may also use the same or similar pathways for signaling (Gilbertson and Khan, 2014, Prog Lipid Res 53:82-92). It is well established that certain diets may influence IBD (Shouval and Rufo, 2017, JAMA Pediatr). Whether and how the taste chemosensory pathways contribute to the effects of diet on IBD remain to be determined. An in-depth understanding of the roles and mechanisms of these chemosensory pathways in IBD may lead to novel treatment strategies for the illness.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition for regulating immune cell activation, the composition comprising a modulator of one selected from the group consisting of a taste receptor type 1 (T1R), a taste receptor type 2 (T2R), Gustducin (Gust), Transient receptor potential cation channel subfamily M member 5 (TrpM5) and a combination thereof.

2. The composition of claim 1, wherein the modulator is an inhibitor of one selected from the group consisting of a T1R, a T2R, Gust, TrpM5 and a combination thereof.

3. The composition of claim 1, wherein the modulator is an activator of one selected from the group consisting of a T1R, a T2R, Gust, TrpM5 and a combination thereof.

4. The composition of claim 1, wherein the modulator is at least one of the group consisting of a chemical compound, a protein, a peptide, a peptidomemetic, an antibody, a ribozyme, a small molecule chemical compound, a nucleic acid, a vector, an antisense nucleic acid molecule.

5. The composition of claim 1, wherein the T1R is selected from the group consisting of T1R1, T1R2, and T1R3.

6. The composition of claim 5, wherein the T1R is T1R3.

7. The composition of claim 1, wherein the T2R is selected from the group consisting of T2R1, T2R2, T2R3, T2R4, T2R5, T2R6, T2R7, T2R8, T2R9, T2R10, T2R11, T2R12, T2R13, T2R14, T2R15, T2R16, T2R17, T2R18, T2R19, T2R20, T2R21, T2R22, T2R24, T2R25, T2R27, T2R28, T2R29, T2R30, T2R31, T2R32, T2R33, T2R34, T2R35, T2R36, T2R37, T2R38, T2R39, T2R40, T2R41, T2R42, T2R43, T2R45, T2R46, T2R50, T2R52, T2R53, T2R54, T2R55, T2R56, T2R57, T2R58, T2R59, T2R60, T2R62P, T2R63P, T2R64P, T2R23, T2R48, T2R49, T2R26, T2R47, T2R44, and T2R51.

8. A method for treating a disease or disorder associated with abnormal immune cell activation, the method comprising administering a modulator of one selected from the group consisting of a taste receptor type 1 (T1R), a taste receptor type 2 (T2R), Gustducin (Gust), Transient receptor potential cation channel subfamily M member 5 (TrpM5) and a combination thereof to a subject in need thereof.

9. The method of claim 8, wherein the disease or disorder is associated with overactive immune cell activation.

10. The method of claim 9, wherein the modulator is an activator of one selected from the group consisting of a T1R, a T2R, Gust, TrpM5 and a combination thereof.

11. The method of claim 9, wherein the disease or disorder is selected from the group consisting of an inflammatory disease or disorder, an autoimmune disease or disorder, an autoinflammatory disease or disorder, a disease or disorder associated with inflammation, and a disease or disorder associated with immune cell activation.

12. The method of claim 8, wherein the disease or disorder is associated with down-regulated immune cell activation.

13. The method of claim 12, wherein the modulator is an inhibitor of one selected from the group consisting of a T1R, a T2R, Gust, TrpM5 and a combination thereof.

14. The method of claim 12, wherein the disease or disorder is selected from the group consisting of cancer and a chronic infectious disease or disorder.

15. The composition of claim 1, wherein the T1R is selected from the group consisting of T1R1, T1R2, and T1R3.

16. The method of claim 8, wherein the T1R is T1R3.

17. The method of claim 8, wherein the T2R is selected from the group consisting of T2R1, T2R2, T2R3, T2R4, T2R5, T2R6, T2R7, T2R8, T2R9, T2R10, T2R11, T2R12, T2R13, T2R14, T2R15, T2R16, T2R17, T2R18, T2R19, T2R20, T2R21, T2R22, T2R24, T2R25, T2R27, T2R28, T2R29, T2R30, T2R31, T2R32, T2R33, T2R34, T2R35, T2R36, T2R37, T2R38, T2R39, T2R40, T2R41, T2R42, T2R43, T2R45, T2R46, T2R50, T2R52, T2R53, T2R54, T2R55, T2R56, T2R57, T2R58, T2R59, T2R60, T2R62P, T2R63P, T2R64P, T2R23, T2R48, T2R49, T2R26, T2R47, T2R44, and T2R51.

18. A method of regulating immune cell activation and/or proliferation, the method comprising administering a modulator of one selected from the group consisting of a taste receptor type 1 (T1R), a taste receptor type 2 (T2R), Gustducin (Gust), Transient receptor potential cation channel subfamily M member 5 (TrpM5) and a combination thereof to a subject in need thereof.

19. The method of claim 18, wherein the method decreases immune cell activation and/or proliferation, and wherein the modulator is an activator of one selected from the group consisting of a T1R, a T2R, Gust, TrpM5 and a combination thereof.

20. The method of claim 18, wherein the method increases immune cell activation and/or proliferation, and wherein the modulator is an inhibitor of one selected from the group consisting of a T1R, a T2R, Gust, TrpM5 and a combination thereof.
